(19) United States
(12) Patent Application Publication
(10) Pub. No.: US 2025/0011473 A1
(43) Pub. Date: Jan. 9, 2025

(54) ANTI-IgE ANTIBODIES

(71) Applicant: UCB BIOPHARMA SRL, Brussels (BE)

(72) Inventors: Ralph ADAMS, Slough (GB); Thomas Allen CESKA, Slough (GB); Anna Marie DAVIES, London (GB); Alistair James HENRY, Slough (GB); Xiaofeng LIU, Slough (GB); James Michael MCDONNELL, London (GB); Brian John SUTTON, London (GB); Marta Katarzyna WESTWOOD, Slough (GB)

(21) Appl. No.: 18/755,124

(22) Filed: Jun. 26, 2024

Related U.S. Application Data
(63) Continuation of application No. 17/541,932, filed on Dec. 3, 2021, now Pat. No. 12,054,559.
Application No. 17/541,932 is a division of application No. 16/307,454, filed on Dec. 5, 2018, now Pat. No. 11,214,627.

(30) Foreign Application Priority Data
Jun. 10, 2016 (GB) 1610198.2
Feb. 15, 2017 (GB) 1702435.7

(51) Int. Cl. C07K 16/42; A61P 37/08; C07K 14/735; C07K 16/28

(52) U.S. Cl. CPC C07K 16/4291; A61P 37/08; C07K 14/70535; C07K 16/2851; C07K 2317/565; C07K 2317/92

(57) ABSTRACT

The present invention relates to the area of improved anti-IgE antibodies and antigen binding agents, and compositions thereof, which target IgE, for instance: for use in treating disorders caused by IgE (such as allergic responses, or certain autoimmune responses); and, in particular, disorders caused by the interaction of IgE with the FcεRI receptor. In particular, this invention relates to improved anti-IgE antibodies and antigen binding agents related to novel mutants of omalizumab (Xolair®). The improved anti-IgE antibodies and antigen binding agents of the invention may have improved affinity for IgE and/or an improved interaction with the Cε2 domain of IgE and/or an improved modified epitope on IgE (for instance further involving the Cε2 domain of IgE) and/or the ability to disassociate IgE from the FcεRI receptor for instance at pharmaceutically-relevant concentrations. In one aspect, improved or novel treatments for IgE mediated disorders are disclosed in which IgE is targeted (for instance free IgE and/or IgE complexed with the FcεRI receptor).

### REFERENCE TO AN ELECTRONIC SEQUENCE LISTING

The contents of the electronic sequence listing (0089-0022US1_SL.txt; Size: 123,000 bytes; and Date of Creation Nov. 13, 2018) is herein incorporated by reference in its entirety.

### FIELD OF THE INVENTION

This invention is in the area of improved anti-IgE antibodies and antigen binding agents, and compositions thereof, which target IgE, for instance for use in treating disorders caused by IgE (such as allergic responses, or certain autoimmune responses), and in particular disorders caused by the interaction of IgE with the FcεRI receptor. In particular, this invention relates to improved anti-IgE antibodies and antigen binding agents related to novel mutants of omalizumab (Xolair®). The improved anti-IgE antibodies and antigen binding agents of the invention may have improved affinity for IgE and/or an improved interaction with the Cε2 domain of IgE and/or an improved modified epitope on IgE (for instance further involving the Cε2 domain of IgE) and/or the ability to disassociate IgE from the FcεRI receptor at pharmaceutically-relevant concentrations. In one aspect, improved or novel treatments for IgE mediated disorders are disclosed in which IgE is targeted (for instance free IgE and/or IgE complexed with the FcεRI receptor).

### BACKGROUND OF THE INVENTION

IgE is a member of the immunoglobulin family that mediates allergic responses such as asthma, food allergies, type 1 hypersensitivity and the familiar sinus inflammation suffered on a widespread basis. IgE is secreted by, and expressed on the surface of, B-cells. IgE synthesized by B-cells is anchored in the B-cell membrane by a transmembrane domain linked to the mature IgE sequence by a short membrane binding region. IgE also is bound to B-cells (and monocytes, eosinophils and platelets) through its Fc region to a low affinity IgE receptor (FcεRII). Upon exposure of a mammal to an allergen, B-cells are clonally amplified which synthesize IgE that binds the allergen. This IgE in turn is released into the circulation by the B-cells where it is bound by B-cells (through FcεRII) and by mast cells and basophils through the so-called high affinity receptor (FcεRI) found on the surface of the mast cells ad basophils. Such mast cells and basophils are thereby sensitized for allergen. The next exposure to the allergen cross-links the FcεRI on these cells and thus activate their release of histamine and other factors which are responsible for clinical hypersensitivity and anaphylaxis.

Omalizumab (Xolair®) is a recombinant DNA-derived humanized IgG1κ monoclonal antibody that selectively binds to human immunoglobulin E (IgE) [the Cε3 domain]. The antibody has a molecular weight of approximately 149 kD. Xolair® is produced by a Chinese hamster ovary cell suspension culture in a nutrient medium containing the antibiotic gentamicin. Xolair® is a sterile, white, preservative-free, lyophilized powder contained in a single-use vial that is reconstituted with Sterile Water for Injection (SWFI), USP, (or, alternatively, as a liquid formulation in a sterile syringe) and administered as a subcutaneous (SC) injection [see EP602126 (and SPC/GB06/005 based thereon); WO93/04173; U.S. Pat. No. 6,267,958 (and the Xolair® PTE based on this patent); WO97/04807; WO97/04801; Presta et al. (1993) J. Immunol. 151:2623-2632].

Omalizumab is presently indicated for the treatment of moderate to severe persistent asthma in patients with a positive skin test or in vitro reactivity to a perennial aeroallergen and symptoms that are inadequately controlled by inhaled corticosteroids (from Xolair® Prescribing Information).

Problems exist with omalizumab in that: 1) it targets free IgE but does not (or does not efficiently) target the pathogenic species of the IgE/FcεRI complex at pharmaceutically-relevant doses; 2) possibly due to the pathogenic species of the IgE/FcεRI complex not being targeted, it takes “at least 12-16 weeks for Xolair treatment to show effectiveness” (Xolair® 150 mg solution—Summary of Product Characteristics 2014)—or indeed to establish whether Xolair® will work for a particular patient or whether a different treatment is necessitated; 3) it should not be for patients with high levels of IgE (for instance because the pathogenic species of the IgE/FcεRI complex is not targeted and does not dissipate with time given the high levels of free IgE in the patient); 4) “Type I local or systemic reactions, including anaphylaxis and anaphylactic shock, may occur when taking omalizumab” (Xolair® 150 mg solution-Summary of Product Characteristics 2014); 5) its affinity for IgE is not particular good (approximately 2 nM).

It is an object of the present invention to identify novel antibodies to ameliorate one or more of these problems.

A further object is to identify antibodies against novel epitopes (with an increased IgE Cε2 interaction compared with omalizumab), and/or antibodies based on novel mutants of omalizumab with improved affinity and/or improved ability to disassociate the IgE/FcεRI complex.

A still further object of the invention to identify new compounds, methods, and compositions for the treatment of disorders associated with IgE, in particular disorders associated with the complex of IgE/FcεRI, for instance allergic disorders.

### SUMMARY OF THE INVENTION

In one aspect of the invention an anti-IgE antibody, or antigen binding agent, is provided which contacts an epitope comprising residues T373, W374, S375, R376, A377, S378, G379, P381, Q417, C418, R419, T421, P426, R427, A428 of a Cε3 domain and residues D278 and T281 of a Cε2 domain of human IgE. In further embodiments the epitope may further comprise one or more of residues K380 and/or M430 of the Cε3 domain of human IgE and/or one or more of residues D276, V277, L279, S280, A282 and/or T298 of the Cε2 domain of human IgE.

The invention is based on the observations of the crystal structure of Example 1 which, for the first time, shows the interaction of an improved antibody (based on omalizumab) with IgE-Fc where significant interactions were observed with the IgE Cε2 domain in the region of mutation. This may result in improved functional characteristics of the anti-IgE antibody, or antigen binding agent, relative to omalizumab and/or omalizumab Fab. For instance, the anti-IgE antibody, or antigen binding agent, may be capable of disassociating human IgE from FcεRI at concentrations (or peak serum concentrations) of less than 7, 3, 1, 0.66, 0.5 or 0.3 μM (for instance as carried out by the method described in Example 2). For instance, the anti-IgE antibody, or antigen binding agent, may have an improved/stronger affinity (lower K_{D})) for human IgE (for instance using IgE-Fc) (for example as carried out by the method described in Example 6) relative to omalizumab and/or omalizumab Fab; and/or an improved ability to disassociate the IgE/FcεRI complex (for instance as determined by the method described in Example 2), relative to omalizumab and/or omalizumab Fab; and/or a capability of disassociating human IgE from FcεRI at concentrations (or peak serum concentrations) lower than for omalizumab and/or omalizumab Fab (for instance as determined by the method described in Example 2). By an improved K_{D} it is meant at least 5, 10, 20, 30, 40, or 50% lower than that of omalizumab and/or omalizumab Fab. The K_{D} of the anti-IgE antibody, or antigen binding agent, of the invention may be less than 2, 1.9, 1.8, 1.7, 1.6, 1.5, 1.4, 1.3, 1.2, 1.1, 1.0, 0.9, 0.8, 0.7, 0.6, 0.5, 0.4, or 0.3 nM. By an improved ability or capability of disassociating human IgE from FcεRI it is meant at least 5, 10, 20, 30, 40, 50, or 100% improved over omalizumab and/or omalizumab Fab (for instance when measuring the % disassociation and/or apparent disassociation rate of the IgE/FcεRI complex as described in Examples 2 and 7), and/or the achievement of disassociation at a concentration where omalizumab and/or omalizumab Fab does not achieve disassociation.

For the avoidance of doubt, the anti-IgE antibody, or antigen binding agent, of the invention is not omalizumab or omalizumab Fab.

In one embodiment, the epitope is determined crystallographically (for Example as described in Example 1) by determining IgE residues within 4 or 5 Å of the anti-IgE antibody, or antigen binding agent, in a crystal structure of complexed IgE-Fc/anti-IgE antibody, or antigen binding agent. The IgE-Fc used may be as that of Seq. ID No. 108 (with the additional N265Q & N371Q mutations).

In one embodiment, the anti-IgE antibody, or antigen binding agent, at a particular binding site, contacts the epitope wherein the Cε3 domain and the Cε2 domain parts of the epitope are on different chains of the human IgE. IgE has two chains in the Fc domain each with a Cε3 domain and a Cε2 domain.

In one embodiment, the anti-IgE antibody, or antigen binding agent, at a particular binding site, contacts the epitope wherein the Cε3 domain and the Cε2 domain parts of the epitope are on the same chain of the human IgE.

For the avoidance of doubt, two anti-IgE antibodies, or antigen binding agents, of the invention may bind to human IgE, but only one of these needs interact with the epitope of the invention comprising Cε3 and Cε2 domains (the other may only interact with the other Cε3 domain for instance).

In one embodiment (optionally further adopting the features of the first aspect of the invention) the anti-IgE antibody, or antigen binding agent, is specific for said epitope comprising residues T373, W374, S375, R376, A377, S378, G379, P381, Q417, C418, R419, T421, P426, R427, A428 of a Cε3 domain and residues D278 and T281 of a Cε2 domain of human IgE. Optionally, said the epitope may further comprise one or more of residues K380 and/or M430 of the Cε3 domain of human IgE and/or one or more of residues D276, V277, L279, S280, A282 and/or T298 of the Cε2 domain of human IgE. For the avoidance of doubt, the anti-IgE antibody, or antigen binding agent is specific for said epitope if it recognizes and binds to the specific human IgE structure comprising said epitope rather than to human IgE generally.

In a further aspect (optionally further adopting the features of the first aspect of the invention) there is provided an anti-IgE antibody, or antigen binding agent, comprising a heavy chain variable region comprising a complementarity determining region, CDR-H3, with an amino acid sequence which is Seq. ID No. 18, and a light chain variable region comprising a complementarity determining region, CDR-L1, with an amino acid sequence which is Seq. ID No. 29, wherein the light chain variable region further comprises a framework region, FR-L3, with an amino acid sequence selected from Seq. ID No. 32 which has one, two, three, four, five, six, seven or more amino acid substitutions to strengthen the interaction of the anti-IgE antibody, or antigen binding agent, with the Cε2 domain of human IgE.

In a further aspect (optionally further adopting the features of the previous aspects of the invention) there is provided an anti-IgE antibody, or antigen binding agent, comprising a heavy chain variable region comprising a complementarity determining region, CDR-H3, with an amino acid sequence which is Seq. ID No. 18, and a light chain variable region comprising a complementarity determining region, CDR-L1, with an amino acid sequence which is Seq. ID No. 29, wherein the light chain variable region further comprises a framework region, FR-L1, with an amino acid sequence which is Seq. ID No. 28 which has one, two, three, four, five, six, seven or more amino acid substitutions to strengthen the interaction of the anti-IgE antibody, or antigen binding agent, with the Cε2 domain of human IgE.

Where the CDR-H3 and CDR-L1 regions anchor and orientate the anti-IgE antibody, or antigen binding agent, on IgE Cε3 region (as per omalizumab), the change(s) to the FR-L3 and/or FR-L1 sequences allow a stronger interaction with the Cε2 domain of human IgE. The stronger interaction of the mutant relative to omalizumab or omalizumab Fab may be assessed through affinity measurements [lower K_{D})](for instance as carried out by the method described in Example 6) and/or the characteristic of improved disassociation of the IgE/FcεRI complex (for instance as determined by the method described in Example 2).

The stronger interaction of the anti-IgE antibody, or antigen binding agent, with the Cε2 domain of human IgE may be characterised by improved functional characteristics of the anti-IgE antibody, or antigen binding agent, relative to omalizumab and/or omalizumab Fab. For instance, the anti-IgE antibody, or antigen binding agent, may be capable of disassociating human IgE from FcεRI at concentrations (or peak serum concentrations) of less than 7, 3, 1, 0.66, 0.5 or 0.3 μM (for instance as carried out by the method described in Example 2). For instance, the anti-IgE antibody, or antigen binding agent, may have an improved/stronger affinity (lower K_{D}) for human IgE (for instance using IgE-Fc) (for example as carried out by the method described in Example 6) relative to omalizumab and/or omalizumab Fab; and/or an improved ability to disassociate the IgE/FcεRI complex (for instance as determined by the method described in Example 2), relative to omalizumab and/or omalizumab Fab; and/or a capability of disassociating human IgE from FcεRI at concentrations (or peak serum concentrations) lower than for omalizumab and/or omalizumab Fab (for instance as determined by the method described in Example 2). By an improved K_{D} it is meant at least 5, 10, 20, 30, 40, or 50% lower than that of omalizumab and/or omalizumab Fab. The K_{D} of the anti-IgE antibody, or antigen binding agent, of the invention may be less than 2, 1.9, 1.8, 1.7, 1.6, 1.5, 1.4, 1.3, 1.2, 1.1, 1.0, 0.9, 0.8, 0.7, 0.6, 0.5, 0.4, or 0.3 nM. By an improved ability or capability of disassociating human IgE from FcεRI it is meant at least 5, 10, 20, 30, 40, 50, or 100% improved over omalizumab and/or omalizumab Fab (for instance when measuring the % disassociation and/or apparent disassociation rate of the IgE/FcεRI complex as described in Examples 2 and 7), and/or the achievement of disassociation at a concentration where omalizumab and/or omalizumab Fab does not achieve disassociation.

For the avoidance of doubt, the anti-IgE antibody, or antigen binding agent, of the invention is not omalizumab or omalizumab Fab.

In one embodiment, the FR-L3 region is mutated, with reference to SEQ ID NO; 129, at one or more of position S60, S63, S76, S77, and/or Q79 (Kabat) to one of the other natural amino acids.

For instance, the FR-L3 region may be mutated at position S60 (Kabat) to one of the other natural amino acids, for instance to M, R, K, N, Q or T, in particular M.

For instance, the FR-L3 region may be mutated at position S63 (Kabat) to one of the other natural amino acids, for instance W or Y, in particular Y.

For instance, the FR-L3 region may be mutated at position S76 (Kabat) to one of the other natural amino acids, in particular N.

For instance, the FR-L3 region may be mutated at position S77 (Kabat) to one of the other natural amino acids, for instance R or K, in particular R.

For instance, the FR-L3 region may be mutated at position Q79 (Kabat) to one of the other natural amino acids, for instance R or K, in particular R.

For instance, the FR-L1 region may be mutated, with reference to SEQ ID NO: 20, on G16 and/or R18 (Kabat) to one of the other natural amino acids.

In certain embodiments, the amino acid sequence of the mutated FR-L3 region of the anti-IgE antibody, or antigen binding agent, is selected from Seq. ID No. 43-49, 60-83, 131 or 138.

In a further embodiment, the FR-L3 region is further mutated, with reference to SEQ ID NO: 129, at position S67 (Kabat) to one of the other natural amino acids to improve its affinity (lower K_{D}) for human IgE. In this case the mutation may be strengthening the interaction of the anti-IgE antibody, or antigen binding agent, for the Cε3 domain of IgE. For instance, the FR-L3 region may be mutated at position S67 (Kabat) to M (in particular), E, or D. In certain embodiments, the amino acid sequence of the mutated FR-L3 region of the anti-IgE antibody, or antigen binding agent, is selected from Seq. ID No. 53-59, 84-107, 131 or 138.

The anti-IgE antibody, or antigen binding agent, of the invention may have the light chain variable region further comprising a complementarity determining region, CDR-L2, with an amino acid sequence which is Seq. ID No. 31.

In one embodiment, the CDR-L2 region is mutated at position S52 (Kabat) to one of the other natural amino acids to improve its affinity (lower K_{D}) for human IgE. In this case the mutation may be strengthening the interaction of the anti-IgE antibody, or antigen binding agent, for the Cε3 domain of IgE. For instance, the CDR-L2 region may be mutated, with reference to SEQ ID NO: 129, at position S52 (Kabat) to D (in particular), E, Q or R. In certain embodiments the amino acid sequence of the mutated CDR-L2 region is selected from Seq. ID No. 50 or Seq. ID No. 51.

The anti-IgE antibody, or antigen binding agent, of the invention may have the heavy chain variable region further comprising a complementarity determining region, CDR-H1, with an amino acid sequence which is Seq. ID No. 14.

The anti-IgE antibody, or antigen binding agent, of the invention may have the heavy chain variable region further comprising a complementarity determining region, CDR-H2, with an amino acid sequence which is Seq. ID No. 16.

The anti-IgE antibody, or antigen binding agent, of the invention may have the light chain variable region further comprising a complementarity determining region, CDR-L3, with an amino acid sequence which is Seq. ID No. 33.

The anti-IgE antibody, or antigen binding agent, of the invention may have the heavy chain variable region further comprising a framework region, FR-H1, with an amino acid sequence which is Seq. ID No. 13.

The anti-IgE antibody, or antigen binding agent, of the invention may have the heavy chain variable region further comprising a framework region, FR-H2, with an amino acid sequence which is Seq. ID No. 15.

The anti-IgE antibody, or antigen binding agent, of the invention may have the heavy chain variable region further comprising a framework region, FR-H3, with an amino acid sequence which is Seq. ID No. 17.

The anti-IgE antibody, or antigen binding agent, of the invention may have the heavy chain variable region further comprising a framework region, FR-H4, with an amino acid sequence which is Seq. ID No. 19.

The anti-IgE antibody, or antigen binding agent, of the invention may have the light chain variable region further comprising a framework region, FR-L2, with an amino acid sequence which is Seq. ID No. 30.

The anti-IgE antibody, or antigen binding agent, of the invention may have the light chain variable region further comprising a framework region, FR-L4, with an amino acid sequence which is Seq. ID No. 34.

The anti-IgE antibody, or antigen binding agent, of the invention may have the light chain variable region, VL, having an amino acid sequence selected from Seq. ID No. 35, Seq. ID No. 132 or Seq ID No. 134 or Seq. ID No. 141 or Seq ID No. 144, or Seq ID No. 145 or Seq ID No. 158 or Seq ID No. 159.

The anti-IgE antibody, or antigen binding agent, of the invention may have the heavy chain variable region, VH, having an amino acid sequence which is Seq. ID No. 1.

The anti-IgE antibody, or antigen binding agent, of the invention may further comprise a light chain constant region.

The anti-IgE antibody, or antigen binding agent, of the invention may have a light chain constant region which is a kappa constant region.

The anti-IgE antibody, or antigen binding agent, of the invention may have the light chain constant region having a mutation L154P (Kabat).

The anti-IgE antibody, or antigen binding agent, of the invention may have the light chain variable region and light chain constant region, VL-CL, having an amino acid sequence selected from Seq. ID No. 39, or Seq. ID No. 41, or Seq. ID No. 117, or Seq. ID No. 119, or Seq. ID No. 125, or Seq. ID No. 127, or Seq. ID No. 136 or Seq. ID No. 143, optionally comprising a signal sequence which has an amino acid sequence which is Seq ID No. 160.

The anti-IgE antibody, or antigen binding agent, of the invention may further comprise a heavy chain constant region, CH1.

The anti-IgE antibody, or antigen binding agent, of the invention may have the heavy chain variable region and heavy chain constant region, VH-CH1, having an amino acid sequence which is Seq. ID No. 5.

The anti-IgE antibody, or antigen binding agent, of the invention may further comprise a heavy chain Fc region, Fc.

The anti-IgE antibody, or antigen binding agent, of the invention may have an Fc which is from human IgG1 or human IgG4.

The anti-IgE antibody, or antigen binding agent, of the invention may have the heavy chain variable region, heavy chain constant region and heavy chain Fc region, VH-CH1-Fc, having an amino acid sequence which is Seq. ID No. 9.

In a further aspect of the invention there is provided an anti-IgE antibody, or antigen binding agent, comprising a heavy chain variable region comprising a complementarity determining region, CDR-H3, with an amino acid sequence which is Seq. ID No. 18, and a light chain variable region comprising a complementarity determining region, CDR-L1, with an amino acid sequence which is Seq. ID No. 29, wherein:
a. the light chain variable region further comprises a framework region, FR-L3, with an amino acid sequence which is Seq. ID No. 32, wherein the FR-L3 region is mutated, with reference to SEQ ID NO: 129, at position S67 (Kabat) to one of the other natural amino acids to improve the affinity (lower K_{D}) of the anti-IgE antibody, or antigen binding agent, for human IgE; and/or
b. the light chain variable region further comprises a complementarity determining region, CDR-L2, with an amino acid sequence which is Seq. ID No. 31, wherein the CDR-L2 region is mutated, with reference to SEQ ID NO: 129, at position S52 (Kabat) to one of the other natural amino acids to improve the affinity (lower K_{D}) of the anti-IgE antibody, or antigen binding agent, for human IgE.

The present inventors, have found herein that either or both of these mutations may surprisingly improve the affinity (improved or lower K_{D}) of an anti-IgE antibody, or antigen binding agent, based on omalizumab or omalizumab Fab, for human IgE (for instance using IgE-Fc) (for example as carried out by the method described in Example 6). In particular, the improvement in affinity is relative to omalizumab and/or omalizumab Fab. The mutations may improve the interaction with the Cε3 domain of IgE. By an improved or lower K_{D} it is meant at least 5, 10, 20, 30, 40, or 50% lower than that of omalizumab and/or omalizumab Fab. The K_{D} of the anti-IgE antibody, or antigen binding agent, of the invention may be less than 2, 1.9, 1.8, 1.7, 1.6, 1.5, 1.4, 1.3, 1.2, 1.1, 1.0, 0.9, 0.8, 0.7, 0.6, 0.5, 0.4, or 0.3 nM.

For instance, the FR-L3 region may be mutated, with reference to SEQ ID NO: 129, at position S67 (Kabat) to M (in particular), E, or D.

In certain embodiments, the amino acid sequence of the mutated FR-L3 region of the anti-IgE antibody, or antigen binding agent, is selected from Seq. ID No. 52-59, 84-107, 131 or 138.

For instance, the CDR-L2 region may be mutated, with reference to SEQ ID NO: 129, at position S52 (Kabat) to D (in particular), E, Q or R.

In certain embodiments, the amino acid sequence of the mutated CDR-L2 region of the anti-IgE antibody, or antigen binding agent, is selected from Seq. ID No. 50 (in particular) or Seq. ID No. 51.

The anti-IgE antibody, or antigen binding agent, may have the heavy chain variable region further comprising a complementarity determining region, CDR-H1, with an amino acid sequence which is Seq. ID No. 14.

The anti-IgE antibody, or antigen binding agent, may have the heavy chain variable region further comprising a complementarity determining region, CDR-H2, with an amino acid sequence which is Seq. ID No. 16.

The anti-IgE antibody, or antigen binding agent, may have the light chain variable region further comprising a complementarity determining region, CDR-L3, with an amino acid sequence which is Seq. ID No. 33.

The anti-IgE antibody, or antigen binding agent, may have the heavy chain variable region further comprising a framework region, FR-H1, with an amino acid sequence which is Seq. ID No. 13.

The anti-IgE antibody, or antigen binding agent, may have the heavy chain variable region further comprising a framework region, FR-H2, with an amino acid sequence which is Seq. ID No. 15.

The anti-IgE antibody, or antigen binding agent, may have the heavy chain variable region further comprising a framework region, FR-H3, with an amino acid sequence which is Seq. ID No. 17.

The anti-IgE antibody, or antigen binding agent, may have the heavy chain variable region further comprising a framework region, FR-H4, with an amino acid sequence which is Seq. ID No. 19.

The anti-IgE antibody, or antigen binding agent, may have the light chain variable region further comprising a framework region, FR-L2, with an amino acid sequence which is Seq. ID No. 30.

The anti-IgE antibody, or antigen binding agent, may have the light chain variable region further comprising a framework region, FR-L4, with an amino acid sequence which is Seq. ID No. 34.

The anti-IgE antibody, or antigen binding agent, may have the light chain variable region, VL, comprising consecutive FR-L1, CDR-L1, FR-L2, CDR-L2, FR-L3, CDR-L3, and FR-L4 regions, and having an amino acid sequence which is Seq. ID No. 20, except that the CDR-L2 region has an amino acid sequence selected from Seq. ID No. 50 (in particular) or Seq. ID No. 51.

The anti-IgE antibody, or antigen binding agent, may have the light chain variable region, VL, comprising consecutive FR-L1, CDR-L1, FR-L2, CDR-L2, FR-L3, CDR-L3, and FR-L4 regions, and having an amino acid sequence which is Seq. ID No. 20, except that the FR-L3 region has an amino acid sequence which is Seq. ID No. 52.

The anti-IgE antibody, or antigen binding agent, may have the light chain variable region, VL, comprising consecutive FR-L1, CDR-L1, FR-L2, CDR-L2, FR-L3, CDR-L3, and FR-L4 regions, and having an amino acid sequence which is Seq. ID No. 20, except that the CDR-L2 region has an amino acid sequence selected from Seq. ID No. 50 (in particular) or Seq. ID No. 51, and the FR-L3 region has an amino acid sequence which is selected from Seq. ID No. 52, 131 or 138.

The anti-IgE antibody, or antigen binding agent, of the invention may have the heavy chain variable region, VH, having an amino acid sequence which is Seq. ID No. 1.

The anti-IgE antibody, or antigen binding agent, may further comprise a light chain constant region.

The anti-IgE antibody, or antigen binding agent, may have a light chain constant region which is a kappa constant region.

The anti-IgE antibody, or antigen binding agent, may have the light chain variable region and light chain constant region, VL-CL, having an amino acid sequence which is Seq. ID No. 24, except that the CDR-L2 region has an amino acid sequence selected from Seq. ID No. 50 (in particular) or Seq. ID No. 51.

The anti-IgE antibody, or antigen binding agent, may have the light chain variable region and light chain constant region, VL-CL, having an amino acid sequence which is Seq. ID No. 24, except that the FR-L3 region has an amino acid sequence which is Seq. ID No. 52.

The anti-IgE antibody, or antigen binding agent, may have the light chain variable region and light chain constant region, VL-CL, having an amino acid sequence which is Seq. ID No. 24, except that the CDR-L2 region has an amino acid sequence selected from Seq. ID No. 50 (in particular) or Seq. ID No. 51, and the FR-L3 region has an amino acid sequence which is selected from Seq. ID No. 52, 131 or 138.

In a further aspect, the invention provides for an anti-IgE antibody, or antigen binding agent, comprising a heavy chain variable region and a light chain variable region, wherein:
a. the heavy chain variable region comprises a CDR-H1 with an amino acid sequence which is Seq ID No. 14, a CDR-H2 with an amino acid sequence which is Seq ID No. 16 and a CDR-H3 with an amino acid sequence which is Seq ID No. 18 and the light chain variable region comprises CDR-L1 with an amino acid sequence which is Seq ID No. 29, a CDR-L2 with an amino acid sequence which is Seq ID No. 50, a CDR-L3 with an amino acid sequence which is Seq ID No. 33 and a framework region FW-L3 with an amino acid sequence which is Seq ID No. 131 or 138; or
b. the heavy chain variable region comprises an amino acid sequence which is Seq ID No. 1 and the light chain variable region comprises an amino acid sequence selected from Seq ID No. 132 or 139.

In one embodiment, the anti-IgE antibody, or antigen binding agent may further comprise a light chain constant region, wherein the light chain variable region and the light chain constant region VL-CL have an amino acid sequence selected from Seq ID No. 136 or 143, optionally comprising a signal sequence which has an amino acid sequence which is Seq ID No. 160.

The anti-IgE antibody, or antigen binding agent, in all embodiments as described herein may further comprise a heavy chain constant region, CH1.

The anti-IgE antibody, or antigen binding agent, may have the heavy chain variable region and heavy chain constant region, VH-CH1, having an amino acid sequence which is Seq. ID No. 5.

The anti-IgE antibody, or antigen binding agent, may further comprise a heavy chain Fc region, Fc.

The anti-IgE antibody, or antigen binding agent, may have the Fc from human IgG1 or human IgG4.

The anti-IgE antibody, or antigen binding agent, may have the heavy chain variable region, heavy chain constant region and heavy chain Fc region, VH-CH1-Fc, having an amino acid sequence which is Seq. ID No. 9.

The anti-IgE antibody, or antigen binding agent, of all aspects of the invention may be selected from the group consisting of: a complete antibody molecule having full length heavy and light chains, or a fragment thereof.

The anti-IgE antibody, or antigen binding agent, of the invention may be elected from the group consisting of: a Fab fragment, modified Fab′ fragment, Fab′ fragment, F (ab′)₂ fragment, Fv, scFv, scab, a diabody, bispecific antibody, triabody, FabFv, Fab-Fv-Fv, tribody, or a (Fab-Fv) 2-Fc. Without being bound by theory, an anti-IgE antibody, or antigen binding agent, of the invention may have less anaphylaxis risk associated with it if it has only one rather than multiple anti-IgE antigen binding sites.

In one embodiment, the anti-IgE antibody is a Fab fragment linked directly or via a linker to a scFv that binds to a serum carrier protein, such as human serum albumin.

In one embodiment the scFv may comprise a heavy chain variable region and a light chain variable region, preferably linked via a linker having Seq. ID No. 151, wherein the heavy chain variable region comprises a CDR-H1 with an amino acid sequence which is Seq ID No. 152, a CDR-H2 with an amino acid sequence which is Seq ID No. 153 and a CDR-H3 with an amino acid sequence which is Seq ID No. 154 and the light chain variable region comprises CDR-L1 with an amino acid sequence which is Seq ID No. 155, a CDR-L2 with an amino acid sequence which is Seq ID No. 156, a CDR-L3 with an amino acid sequence which is Seq ID No. 157.

In one embodiment the scFv has an amino acid sequence which is Seq ID No. 150. In one preferred embodiment, the Fab fragment comprises a heavy chain variable region and a light chain variable region, wherein:
a. the heavy chain variable region comprises a CDR-H1 with an amino acid sequence which is Seq ID No. 14, a CDR-H2 with an amino acid sequence which is Seq ID No. 16 and a CDR-H3 with an amino acid sequence which is Seq ID No. 18 and the light chain variable region comprises CDR-L1 with an amino acid sequence which is Seq ID No. 29, a CDR-L2 with an amino acid sequence which is Seq ID No. 50, a CDR-L3 with an amino acid sequence which is Seq ID No. 33 and a framework region FW-L3 with an amino acid sequence which is Seq ID No. 131 or 138; or
b. the heavy chain variable region comprises an amino acid sequence which is Seq ID No. 1 and the light chain variable region comprises an amino acid sequence selected from Seq ID No. 132 or 139.

In another embodiment, the Fab fragment further comprises a heavy chain constant region and a light chain constant region, wherein the heavy chain variable region and the heavy chain constant region VL-CH1 has an amino acid sequence which is Seq ID No. 5 and wherein the light chain variable region and the light chain constant region VL-CL has an amino acid sequence selected from Seq ID No. 136 or 143, optionally comprising a signal sequence which has an amino acid sequence which is Seq ID No. 160.

In another embodiment scFv is linked to the CH1 of the Fab fragment via a linker having amino acid sequence which is Seq ID No. 149.

In one embodiment, the heavy chain variable region and the heavy chain constant region, the linker and the scFv has an amino acid sequence which is Seq ID No. 147, optionally comprising a signal sequence which has an amino acid sequence which is Seq ID No. 160.

In one other embodiment the heavy chain of the Fab fragment linked to the scFv with Seq. ID No. 147 is paired with a light chain variable and constant region which has Seq ID No. 136 or 143.

The anti-IgE antibody, or antigen binding agent, of the invention may have an effector or a reporter molecule attached to it.

The anti-IgE antibody, or antigen binding agent, of the invention may be glycosylated (for instance within the Fc domain) and/or may be conjugated to a polymer selected from starch, albumin, and polyethylene glycol (PEG). In one embodiment, conjugated PEG may have a molecular weight in the range 5 to 50 kDa.

The anti-IgE antibody, or antigen binding agent, of the invention may be humanized.

A further aspect of the invention relates to an isolated DNA sequence encoding the heavy and/or light chain(s) of the anti-IgE antibody, or antigen binding agent, of the invention. Further provided is a cloning or expression vector comprising one or more DNA sequences of the invention. For instance, a cloning or expression vector may comprise one or more DNA sequences selected from Seq. ID No. 36, Seq. ID No. 38, Seq. ID No. 40, or Seq. ID No. 42, or Seq. ID No. 133, or Seq ID No. 135, or Seq ID No. 137, or Seq ID No. 140, or Seq. ID No. 142, or Seq ID No. 144 and, optionally, may further comprise one or more DNA sequences selected from Seq. ID No. 2, Seq. ID No. 4, Seq. ID No. 6, Seq. ID No. 8, Seq. ID No. 10, or Seq. ID No. 12 or Seq. ID No. 148.

A further aspect of the invention is a host cell comprising one or more cloning or expression vectors of the invention. The host cell of the invention may optionally further comprise one or more cloning or expression vectors comprising one or more DNA sequences selected from Seq. ID No. 2, Seq. ID No. 4, Seq. ID No. 6, Seq. ID No. 8, Seq. ID No. 10, or Seq. ID No. 12 or Seq ID No. 148.

A process for the production of the anti-IgE antibody, or antigen binding agent, of the invention is also provided, comprising culturing the host cell of the invention and isolating the anti-IgE antibody, or antigen binding agent.

A further aspect relates to a pharmaceutical composition comprising the anti-IgE antibody, or antigen binding agent, of the invention, in combination with one or more of a pharmaceutically acceptable excipient, diluent or carrier. Suitably, the anti-IgE antibody, or antigen binding agent, of the invention is present at a dose of 50-200, preferably around or exactly 150 mg per mL diluent. In certain embodiments, the excipient comprises one or both of L-arginine, L-histidine. The excipient may separately or in combination comprise Polysorbate 20. The diluent may be water or an aqueous isotonic solution.

The pharmaceutical composition of the invention may be carried within a sterile vial as a powder for reconstitution prior to subcutaneous administration, or within a sterile syringe for its immediate subcutaneous administration.

The pharmaceutical composition of the invention may contain a total dose of anti-IgE antibody, or antigen binding agent, of 75-600 mg—for instance around or exactly 100 or 150 mg.

The pharmaceutical composition of the invention may additionally comprise other active ingredients either contained together with the anti-IgE antibody, or antigen binding agent, or for separate coadministration with the anti-IgE antibody, or antigen binding agent. For instance, the pharmaceutical composition of the invention may be used in the context of allergy-based specific immunotherapy, where the anti-IgE antibody, or antigen binding agent, of the invention is separately coadministered (but may be co-packaged) with an allergen. The pharmaceutical composition of the invention may thus be for use in allergy-based specific immunotherapy, where the patient receives the pharmaceutical composition of the invention 7, 6, 5, 4, 3, 2, or 1 days before (or on the same day) as the therapeutic allergen.

The anti-IgE antibody, or antigen binding agent, or composition, of the invention may be for use as a medicament.

The anti-IgE antibody, or antigen binding agent, or composition, of the invention may be for use in the treatment or prevention of disease.

The anti-IgE antibody, or antigen binding agent, or composition, of the invention may be for use in the treatment or prevention of disorders associated with the complex of human IgE and FcεRI.

The anti-IgE antibody, or antigen binding agent, or composition, of the invention may be for use in the treatment or prevention of disorders through the disassociation of the complex of human IgE and FcεRI and the binding of human IgE by the anti-IgE antibody, or antigen binding agent.

The anti-IgE antibody, or antigen binding agent, or composition, of the invention may be for use in the treatment or prevention of one or more of: allergy; allergic asthma; severe asthma; moderate asthma; chronic spontaneous urticaria; chronic idiopathic urticaria; perennial allergic rhinitis; seasonal allergic rhinitis; acute asthma exacerbations; acute bronchospasm; status asthmaticus; hyper IgE syndrome; allergic bronchopulmonary aspergillosis; prevention of anaphylactic reactions; food allergy; atopic dermatitis; allergic rhinitis; bee venom sensitivity; idiopathic anaphylaxis; peanut allergy; latex allergy; inflammatory skin diseases; urticaria (solar, cold-induced, local heat-induced, and/or delayed pressure-induced); cutaneous mastocytosis; systemic mastocytosis; eosinophil-associated gastrointestinal disorder; bullous pemphigoid; interstitial cystitis; nasal polyps; idiopathic angioedema; or non-allergic asthma.

Further provided is a method for the treatment or prevention of a disease in a human subject, the method comprising administering to the subject an effective amount of the anti-IgE antibody, or antigen binding agent, or a composition of the invention. The method may be for the treatment or prevention of disorders associated with the complex of human IgE and FcεRI. The method of the invention may treat or prevent disease through the disassociation of the complex of human IgE and FcεRI and the binding of human IgE by the anti-IgE antibody, or antigen binding agent of the invention.

The method of the invention may be for the treatment or prevention of one or more of: allergy; allergic asthma; severe asthma; moderate asthma; chronic spontaneous urticaria; chronic idiopathic urticaria; perennial allergic rhinitis; seasonal allergic rhinitis; acute asthma exacerbations; acute bronchospasm; status asthmaticus; hyper IgE syndrome; allergic bronchopulmonary aspergillosis; prevention of anaphylactic reactions; food allergy; atopic dermatitis; allergic rhinitis; bee venom sensitivity; idiopathic anaphylaxis; peanut allergy; latex allergy; inflammatory skin diseases; urticaria (solar, cold-induced, local heat-induced, and/or delayed pressure-induced); cutaneous mastocytosis; systemic mastocytosis; eosinophil-associated gastrointestinal disorder; bullous pemphigoid; interstitial cystitis; nasal polyps; idiopathic angioedema; or non-allergic asthma.

In the present invention, it has been elucidated that an antibody or an antigen binding agent against a first polypetide, which polypetide elicits its physiological response by virtue of binding to a second polypetide (such as a receptor), is capable of binding to both the free and bound first polypetide, stabilising a conformation of such first polypetide. Such stabilised conformation has a binding affinity for the second polypetide weaker than in the absence of the antibody or antigen binding agent hence, triggering faster dissociation of the first polypetide from the second polypetide.

In this respect, the invention provides a further aspect which relates to an antibody or an antigen binding agent, capable of binding free and FcεRI bound human IgE and stabilising a conformation of IgE. When the IgE is in such conformation, it has a binding affinity for FcεRI weaker than in the absence of the antibody or antigen binding agent and wherein the FcεRI bound human IgE dissociates from FcεRI. Optionally, when the IgE is in such conformation, the IgE has a lower binding affinity for omalizumab or a fragment thereof than the antibody or antigen binding agent of the invention. For example, the antibody or antigen binding agent is an antibody as described herein.

In a further aspect, the invention relates to a process for selecting such antibodies or antigen binding agents as described herein. The process comprises:

a. Contacting a test antibody or antigen binding agent with a sample comprising human IgE bound to human FcεRI;
b. Measuring the constant of dissociation of the test antibody or antigen binding agent for dissociation the human IgE from human FcεRI;
c. Comparing the constant of dissociation as measured in step b) with the constant of dissociation of omalizumab or a fragment thereof for dissociating the human IgE from to human FcεRI;
d. Selecting the antibody or antigen binding agent if said antibody or antigen binding agent dissociates the IgE from FcεRI faster than omalizumab or a fragment thereof.

Alternatively, the process for selecting antibodies or antigen binding agents according to the invention comprises:
a. Contacting a test antibody or antigen binding agent with a sample comprising human IgE bound to human FcεRI;
b. Measuring the binding affinity of the test antibody or antigen binding agent for human IgE from human FcεRI;
c. Comparing the binding affinity as measured in step b) with the binding affinity of human IgE for FcεRI;
d. Selecting the antibody or antigen binding agent if said antibody or antigen binding agent has a higher binding affinity for IgE than the IgE for FcεRI.

Optionally, the selected antibodies or antigen binding agents cause the IgE, whilst still bound to FcεRI, to adopt a conformation wherein the IgE in said stabilised conformation may dissociate from FcεRI faster than the IgE bound to FcεRI in the presence of omalizumab or a fragment thereof; and/or may have a binding affinity higher for the antibody or antigen binding agent than for the FcεRI.

In a final aspect, the present invention relates to specific antibodies or antigen binding agent comprising:
a. a heavy chain variable region comprising Seq. ID No.: 1 and a light chain variable region comprising:
   i. Seq. ID No.: 109; or
   ii. Seq. ID No.: 113; or
   iii. Seq. ID No.: 121; or
   iv. Seq. ID No.: 132; or
   v. Seq. ID No.: 139; or
b. Seq. ID No.: 5 and
   i. Seq. ID No.: 24, wherein S77 and S79 are replaced by Q;
   ii. Seq. ID No.: 117 or
   iii. Seq. ID No.: 125; or
   iv. Seq. ID No.: 136; or
   v. Seq. ID No.: 143.

In one embodiment of this last aspect of the invention, the anti-IgE antibody or antigen binding agent contacts or, contacts and is specific for, an epitope comprising, with reference to SEQ ID NO: 108, residues T373, W374, S375, R376, A377, S378, G379, P381, Q417, C418, R419, T421, P426, R427, A428 of a Cε3 domain and residues D278 and T281 of a Cε2 domain of human IgE.

### BRIEF DESCRIPTION OF THE DRAWINGS

References and SEQ IDs are found in the Examples referring to the Figures.

FIG. 1. omalizumab Fab3 contains three point mutations. omalizumab Fab3 is derived from omalizumab, and contains three point mutations distal to the antigen binding CDRs, two in the VL domain framework region (Ser81Arg, Gln83Arg) and one in the Cκ domain (Leu158Pro). The heavy and light chains are colored white and blue, respectively. The mutated residues are colored red, and CRDL1 in green, to indicate the orientation of the Fab.

FIG. 2. Overall structure of IgE-Fc in complex with omalizumab Fab3. (A) omalizumab Fab3 binds to IgE-Fc with 2:1 stoichiometry. Fab1 (green) engages IgE-Fc chain B (pink) exclusively through the Cε3 domain. Fab² (blue) interacts with IgE-Fc chain A (yellow) through the Cε3 domain and forms a minor interaction with the Cε2 domain from IgE-Fc chain B (pink). (B) The two Fabs form a pseudo-symmetric complex about the two-fold axis of the Fcε3-4 region. For clarity, the Cε2 domains are not shown. (C) IgE-Fc is asymmetrically bent in the omalizumab Fab3 complex. The Cε2 domain from chain B (pink) contacts Fab² (blue).

FIG. 3. Interface between omalizumab Fab3 and IgE-Fc. The interface between omalizumab Fab3 Fab² (heavy and light chains colored in green and yellow, respectively) and the Cε3 domain from IgE-Fc (pink) is shown. omalizumab Fab3 and Cε3 domain residue labels are colored blue and black, respectively. The interface comprises hydrogen bonds and van der Waals interactions. A notable feature of the interface is a cation-x interaction between Arg419 (Cε3 domain) and Phe103 (omalizumab Fab3 CDRH3). The Phe103 side chain is mostly buried in a pocket created by Thr373, Trp374, Ser375, Gln417 and Arg419 (Cε3 domain).

FIG. 4. omalizumab Fab3 and DARPin E2_79 bind to an overlapping interface. omalizumab Fab3 and DARPin E2 7920 both bind to the Cε3 domain. IgE-Fc residues which only form part of the omalizumab Fab3 interface are colored orange, while those which only form part of the DARPin E2_79 interface, which includes part of the Cε3-Cε4 linker, are colored in blue. IgE-Fc residues colored in pink, which include Arg419 and Met430, are common to both omalizumab Fab3 and DARPin E2_79 interfaces.

FIG. 5. Conformational flexibility in IgE-Fc. (A) Side view of free IgE-Fc⁸ showing its acute, asymmetric bend. (B) Front view of free IgE-Fc (90° anti-clockwise rotation from the view shown in (A). (C) Side view of IgE-Fc from the omalizumab Fab3 complex, revealing a partially bent conformation. (D) Front view of IgE-Fc in the omalizumab Fab3 complex (90° anti-clockwise rotation from the view shown in (C). (E) Side view of fully extended IgE-Fc captured by an anti-IgE-Fc Fab (omalizumab Fab3)¹⁶. (F) Front view of extended IgE-Fc (90° anti-clockwise rotation from the view shown in (E).

FIG. 6. Conformational flexibility in IgE-Fc. Flexibility of IgE-Fc, and unbending from the bent to a fully extended conformation, was previously explored by molecular dynamics¹⁶. IgE-Fc unbending is represented as a free energy surface, as previously described¹⁶. (A) Extended conformation of IgE-Fc captured in the crystal structure of the omalizumab Fab3/IgE-Fc complex¹⁶. (B) Partially bent IgE-Fc conformation observed in the crystal structure of the omalizumab Fab3/IgE-Fc complex. (C) Bent conformation of free IgE-Fc^{7.8}. The bent conformation of IgE-Fc occupies the lowest energy basin, while the partially bent conformation observed in the omalizumab Fab3/IgE-Fc complex occupies a clearly distinct energy basin (B).

FIG. 7. Disruption of the interaction between IgE-Fc and FcεRI. In the omalizumab Fab3 complex, the Cε3 domains adopt the most open conformation reported thus far for IgE-Fc, which precludes engagement with FcεRIα. The structure of IgE-Fc in complex with FcεRIα⁸ is colored yellow, and the two sub-sites of receptor engagement are indicated. The structure of omalizumab Fab3 in complex with IgE-Fc was superposed on the Ca4 domains, and the Cε3 domains are colored blue. Positions of His424 and Pro426 in the two structures are indicated, to highlight the different positions adopted by the Cε3 domains.

FIG. 8. Disruption of the interaction between IgE-Fc and CD23. (A) Cε3 domain residues which are common to both omalizumab Fab3 and CD23 interfaces are colored pink. (B) Superposition of the Cε3 domains (dark gray) from the omalizumab Fab3/IgE-Fc complex and CD23/Fcε3-4 complex¹¹ reveal clashes between CD23 (yellow) and omalizumab Fab3 (pink).

FIG. 9. Interaction studies of omalizumab Fab3 with IgE-Fc. (A) Binding of omalizumab Fab3 to IgE-Fc captured via a C-terminal His-tag; omalizumab Fab3 was flowed over IgE-Fc at the following concentrations 100 nM (black), 50 nM (red), 25 nM (green), 12.5 nM (blue), 6.2 nM (cyan), 3.1 nM (purple), 1.6 nM (magenta) and 0.8 nM (dark red). Standard double referencing methods were employed³⁶; each concentration was run in duplicate. (B) Binding of the second omalizumab Fab3 binding site was characterized using an SPR sandwich binding experiment. IgE-Fc was captured on an omalizumab Fab3 surface, and then a second omalizumab Fab3 molecule was added to the IgE-Fc/omalizumab Fab3 complex at concentrations of 1000 nM (black), 500 nM (red), 250 nM (green), 125 nM (blue), 62.5 nM (cyan), 31.2 nM (purple), 15.6 nM (magenta), 7.8 nM (dark red) and 0 nM (navy). (C) A comparison of the ability of omalizumab Fab3 to bind to IgE-Fc captured by a C-terminal His-tag (red) and IgE-Fc captured by binding to FcεRIα (blue); a two-fold dilution series was tested for each, with a highest concentration of 1000 nM. The inset shows that omalizumab Fab3 can still bind to the IgE-Fc/FcεRIα complex, but with a low Bₘₐₓ value. (D) Accelerated dissociation of the IgE-Fc/FcεRIα complex mediated by increasing concentrations of omalizumab Fab3. The 1:1 IgE-Fc/FcεRIα complex was first established by capturing IgE-Fc on immobilized FcεRIα and then binding omalizumab Fab3 at the following concentrations: 5000 nM (magenta), 1000 nM (purple), 200 nM (cyan), 40 nM (blue), 8 nM (green), 1.6 nM (red) and 0 nM (black). The inset shows a magnification of the accelerated dissociation process. All concentrations were run in duplicate. All binding experiments were performed at 25° C., except those characterizing the second omalizumab Fab3 binding site (FIG. 4B), which were done at 5° C. to minimize allosteric communication between the two sites.

FIG. 10. Analysis of direct binding, competition experiments and accelerated dissociation. Direct binding was measured for IgE-Fc to immobilized omalizumab Fab3 (A), omalizumab Fab (B), and intact omalizumab (C). Fabs or intact antibody were covalently immobilized at low density using an amine coupling kit (GE Healthcare); IgE-Fc was flowed over these surfaces at a variety of concentrations, using a two-fold dilution series with a highest concentration of 100 nM. All concentrations were run in duplicate. (D) TR-FRET competition binding experiments between omalizumab Fab3 and ay-fusion protein for IgE-Fc. Binding between terbium-labeled ay-fusion protein and Alexa Fluor 647-labeled IgE-Fc was measured with increasing concentrations of unlabeled omalizumab Fab3 as inhibitor: 0 μM (black), 2.5 nM (blue), 5 nM (green), 10 nM (magenta), 20 nM (red). As an inhibitor, omalizumab Fab affects both the apparent K_{D} and Bₘₐₓ of the interaction between IgE-Fc and αγ-fusion protein, indicating some allosteric inhibition properties. (E) Comparison of the accelerated dissociation of the IgE-Fc/sΓcεRIa complex mediated by intact omalizumab (black), omalizumab Fab (red) or omalizumab Fab3 (blue), each at a concentration of 5 μM.

FIG. 11. Representative electron density map. A stereoview of the 2Fₒ-Fₑ electron density map, contoured at 1.1σ, is shown for a portion of the chain A Cε3 domain, and covalently N-linked oligosaccharide moiety at Asn394.

FIG. 12. Biacore sensorgram of the dissociation of IgE-Fc from immobilized sFcεRIα. Dissociation was monitored in the presence of running buffer (solid line) or IgE binding partners (all other sensorgrams). Assay performed as described in Assay Method (1) Example 2.

FIG. 13. Biacore sensorgram of the dissociation of IgE-Fc from immobilized sFcεRIα. Dissociation was monitored in the presence of control supernatant (solid line) or IgE binding partners (all other sensorgrams). Assay performed as described in Assay Method (2) Example 2.

FIG. 14. Analysis of dissociation of Alexa488 labelled IgE-Fc from the surface of RBL-SX38 cells. Measured binding data normalized to 100% at t=0 and dissociation data plotted as the change in proportion of IgE-Fc remaining bound as a function of time.

FIG. 15. Analysis of the effect of therapeutic dosing of wild type omalizumab Fab and omalizumab Fab3 on the 72 hour PCA model.

FIG. 16. Residues 224 to 547 of wild-type human IgE-Fc sequence (as shown in SEQ ID NO: 108) with residues 224 and 547 shown in bold. The numbering is according to Dorrington and Bennich (1978) Immunol. Rev. 41:3-25, whereby the L (Leu; leucine) after L253 is numbered as L235a (boxed) and the subsequent residue is C254. The remaining residues are numbered consecutively with no further additions. The epitope residues are shown with an asterisk (*).

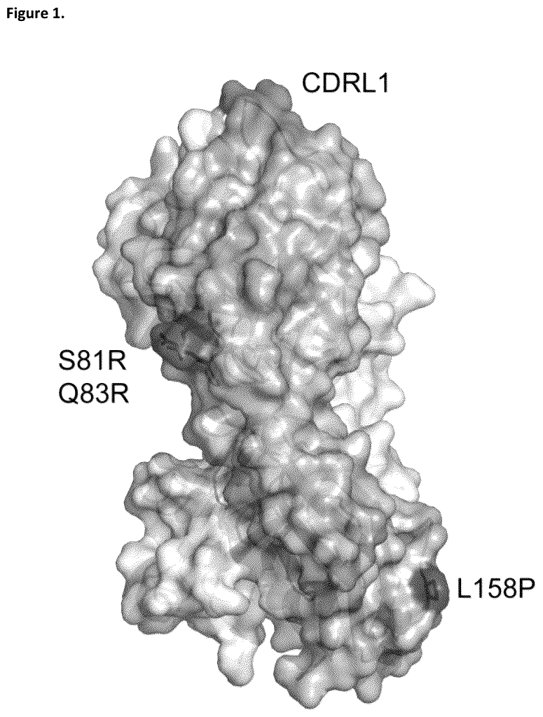

### DETAILED DESCRIPTION OF THE INVENTION

Antibody amino acid numbering herein will either be from the consecutive amino acid sequence of an antibody (for instance omalizumab comprising VH sequence of Seq. ID No. 1 and VL sequence of Seq. ID No. 20 or SEQ ID NO: 129)—the so called “pdb” numbering—or may utilize the common Kabat numbering scheme. Where the common immunoglobulin parts (CDRs—complementarity determining regions, or FRs-framework regions) of a VH or VL sequence are described, they are linked in the standard order (VH=FR-H1.CDR-H1.FR-H2.CDR-H2.FR-H3.CDR-H3.FR-H4; VL=FR-L1.CDR-L1.FR-L2.CDR-L2.FR-L3.CDR-L3.FR-L4). For omalizumab “pdb” numbering of VH (Seq. ID No. 1) parts is: FR-H1 (amino acids 1-25), CDR-H1 (26-36), FR-H2 (37-50), CDR-H2 (51-66), FR-H3 (67-98), CDR-H3 (99-110), FR-H4 (111-121); whereas Kabat numbering is: FR-H1 (amino acids 1-25), CDR-H1 (26-35), FR-H2 (36-49), CDR-H2 (50-65), FR-H3 (66-94), CDR-H3 (95-102), FR-H4 (103-113). For omalizumab “pdb” numbering of VL (Seq. ID No. 20) parts is: FR-L1 (amino acids 1-23), CDR-L1 (24-38), FR-L2 (39-53), CDR-L2 (54-60), FR-L3 (61-92), CDR-L3 (93-101), FR-L4 (102-111); whereas Kabat numbering is: FR-L1 (amino acids 1-23), CDR-L1 (24-34), FR-L2 (35-49), CDR-L2 (50-56), FR-L3 (57-88), CDR-L3 (89-97), FR-L4 (98-107).

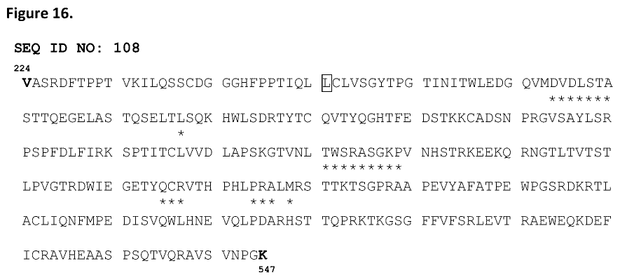

IgE antibody numbering is as reported by Dorrington & Bennich (1978) Immunol. Rev. 41:3-25). Thus, the IgE-Fc polypeptides used in this invention (see Seq. ID No. 108) are from V224-K547 (including a C225A mutation). As shown in FIG. 16, the numbering followed is according to Dorrington and Bennich (1978) Immunol. Rev. 41:3-25 where the L (Leu, leucine) following position 253 is numbered L253a and the remaining residues are numbered consecutively from L253a as C254 etc. In the crystallography experiments the following mutations were also inserted into the IgE-Fc to simplify the glycosylation pattern: N265Q & N371Q. The Cε2 region of IgE-Fc is generally accepted to occupy the sequence S226-D330. Reference to IgE herein may be a reference to human IgE (and vice versa), and may also constitute a reference to IgE-Fc in the context of the assays and methods described herein. The sequence of the Fab arms of the full length human IgE antibody are not included in this description as they are not present in the crystal structures.

Herein reference to “omalizumab” is a reference to the commercially-sold Xolair® product; or to an IgG full-length antibody comprising a heavy chain comprising the VH amino acid sequence which is Seq. ID No. 1, and a light chain comprising the VL amino acid sequence which is Seq. ID No. 20; or to an IgG full-length antibody comprising a heavy chain comprising the VH-CH1 amino acid sequence which is Seq. ID No. 5, and a light chain comprising the VL-CL amino acid sequence which is Seq. ID No. 24; or to an IgG full-length antibody comprising a heavy chain comprising the VH-CH1-Fc amino acid sequence which is Seq. ID No. 9, and a light chain comprising the VL-CL amino acid sequence which is Seq. ID No. 24. Reference to “omalizumab Fab” is a reference to a Fab fragment comprising a heavy chain comprising the VH amino acid sequence which is Seq. ID No. 1, and a light chain comprising the VL amino acid sequence which is Seq. ID No. 20; or (in particular) to a Fab fragment comprising a heavy chain comprising the VH-CH1 amino acid sequence which is Seq. ID No. 5, and a light chain comprising the VL-CL amino acid sequence which is Seq. ID No. 24.

### General Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques may be performed according to manufacturer's specifications, as commonly accomplished in the art or as described herein. The nomenclatures used in connection with, and the laboratory procedures and techniques of, analytical chemistry, synthetic organic chemistry, and medicinal, and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, selected terms are defined below.

The term “host” as used herein, typically refers to a human subject, and in particular where a human or humanized framework is used as an acceptor structure. Where another host is treated, it is understood by those of skill in the art that the antibody or antigen binding agent may need to be tailored to that host to avoid rejection or to make more compatible. It is known how to use the CDRs in the present invention and engineer them into the proper framework or peptide sequence for desired delivery and function for a range of hosts. Other hosts may include other mammals or vertebrate species. The term “host,” therefore, can alternatively refer to animals such as mice, monkeys, dogs, pigs, rabbits, domesticated swine (pigs and hogs), ruminants, equine, poultry, felines, murines, bovines, canines, and the like, where the antibody or antigen binding agent, if necessary is suitably designed for compatibility with the host.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric.

The term “recovering” as used herein, refers to the process of rendering a chemical species such as a polypeptide substantially free of naturally associated components by isolation, e.g., using protein purification techniques well known in the art.

The terms “specific binding” or “specifically binding”, as used herein, in reference to the interaction of an antibody, a protein, or a peptide with a second chemical species, mean that the interaction is dependent upon the presence of a particular structure (e.g., an “antigenic determinant” or “epitope” as defined below) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. Where an epitope of the invention is mentioned herein, the anti-IgE antibody, or antigen binding agent, of the invention is specific for said epitope.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains at least some portion of the epitope binding features of an Ig molecule allowing it to specifically bind to IgE. Such mutant, variant, or derivative antibody formats are known in the art and described below. Non limiting embodiments of which are discussed below. An antibody is said to be “capable of binding” a molecule (or epitope) if it is capable of specifically reacting with the molecule (or epitope) to thereby bind the molecule (or epitope) to the antibody.

A “monoclonal antibody” as used herein is intended to refer to a preparation of antibody molecules, which share a common heavy chain and common light chain amino acid sequence, or any functional fragment, mutant, variant, or derivation thereof which retains at least the light chain epitope binding features of an Ig molecule, in contrast with “polyclonal” antibody preparations that contain a mixture of different antibodies. Monoclonal antibodies can be generated by several known technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497).

In a full-length antibody, each heavy chain is comprised of a heavy chain variable region (abbreviated herein as HCVR or VH) and a heavy chain constant region (CH). The heavy chain constant region is comprised of four domains—either CH1, Hinge, CH2, and CH3 (heavy chains γ, a and 8), or CH1, CH2, CH3, and CH4 (heavy chains μ and ε). Each light chain is comprised of a light chain variable region (abbreviated herein as LCVR or VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antigen binding agent” as used herein, refers to one or more fragments or portions of an antibody that retain the ability to specifically bind to an antigen (e.g., IgE), or synthetic modifications of antibody fragments that retain the desired binding ability to the antigen. It has been shown that the antigen-binding function of an antibody can be performed by fragments or certain portions of a full-length antibody, or modifications thereof. Embodiments include bispecific, dual specific and multi-specific formats which may specifically bind to two or more different antigens or to several epitopes or discontinuous epitope regions of an antigen. Non limiting examples of antigen binding agents include (i) a Fab fragment, a monovalent fragment consisting of the VL, VH, CL, and CH1 domains; (ii) a F (ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; (vi) an isolated complementarity determining region (CDR), (vii) fusions of antibody fragments such as those that are immunoglobulin in character, for example, diabodies, scab, bispecific, triabody, Fab-Fv, Fab-Fv-Fv, tribody, (Fab-Fv) 2-Fc, and (viii) antibody portions such as CDRs or antibody loops grafted onto non-immunoglobulin frameworks such as fibronectin or leucine zippers (see Binz et al. (2005) Nat. Biotech. 23:1257-1268, incorporated herein). Furthermore, although the two domains of the Fv fragment, VL and VH, are coded for by separate genes, they can be joined, using recombinant or other methods, by a synthetic or naturally occurring linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term antigen binding agent. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more of the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain, for example a human IgA, IgD, IgE, IgG or IgM constant domains. Heavy chain and light chain constant domain amino acid sequences are known in the art. Non-limiting examples of Ig heavy chain yl constant region and Ig light chain λ and κ chains are provided for in Tables 8 and 6, respectively.

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F (ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds IgE is substantially free of antibodies that specifically bind antigens other than IgE). An isolated antibody that specifically binds, for example, human IgE may, however, have cross-reactivity to other antigens, such as IgE molecules from other species. Moreover, an isolated antibody may be substantially free of other cellular material and/or chemicals.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs (e.g., CDR3) has been replaced with murine CDR sequences.

The terms “Kabat numbering”, “Kabat definitions” and “Kabat labelling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31-35 (CDR-H1), residues 50-65 (CDR-H2) and residues 95-102 (CDR-H3) according to the Kabat numbering system. However, according to Chothia (Chothia et al., (1987) J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. For the light chain variable region, the hypervariable region ranges from amino acid positions 24 to 34 for CDRL1, amino acid positions 50 to 56 for CDRL2, and amino acid positions 89 to 97 for CDRL3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDRH1, CDRH2 and CDRH3 for the heavy chain CDRs, and CDRL1, CDRL2, and CDRL3 for the light chain CDRs. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia, or a mixture thereof, defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone conformations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1,-L2, and—L3 of light chain and CDR-H1,-H2, and—H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. See, e.g., Shapiro et al., Crit. Rev. Immunol. 22 (3): 183-200 (2002); Marchalonis et al., Adv Exp Med Biol. 484:13-30 (2001). One of the advantages provided by various embodiments of the present invention takes advantage of the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N—or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

The term “humanized antibody” generally refers to antibodies which comprise heavy and light chain variable region sequences from a non-human species (e.g., a rabbit, mouse, etc.) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Another type of humanized antibody is a CDR-grafted antibody, in which at least one non-human CDR is inserted into a human framework. The latter is typically the focus of the present invention.

In particular, the term “humanized antibody” as used herein, is an antibody or a variant, derivative, analog or fragment thereof which immuno-specifically binds to an antigen of interest and which comprises a framework (FR) region having substantially the amino acid sequence of a human antibody and a complementarity determining region (CDR) having substantially the amino acid sequence of a non-human antibody. As used herein, the term “substantially” in the context of a CDR refers to a CDR having an amino acid sequence at least 50, 55, 60, 65, 70, 75 or 80%, preferably at least 85%, at least 90%, at least 95%, at least 98% or at least 99% identical to the amino acid sequence of a non-human antibody CDR. In one embodiment, the humanized antibody has a CDR region having one or more (for example 1, 2, 3 or 4) amino acid substitutions, additions and/or deletions in comparison to the non-human antibody CDR. Further, the non-human CDR can be engineered to be more “human-like” or compatible with the human body, using known techniques. A humanized antibody comprises substantially all of at least one, and typically two, variable domains (Fab, Fab′, F(ab′)2, F (ab′) c, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, and CH3, or CH1, CH2, CH3, and CH4 of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain. Though some of the mutations discussed herein may not be commonly “human”, these are insufficient for the anti-IgE antibody, or antigen binding agent, of the invention not to be “humanized”.

The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond exactly to either the donor antibody or the consensus framework. In a preferred embodiment, such mutations, however, will not be extensive. Usually, at least 50, 55, 60, 65, 70, 75 or 80%, preferably at least 85%, more preferably at least 90%, and most preferably at least 95%, 98% or 99% of the humanized antibody residues will correspond to those of the parental FR and CDR sequences. In one embodiment, one or more (for example 1, 2, 3, 4, 5, 6, 7, 8, 9 or 10) amino acid substitutions, additions and/or deletions may be present in the humanized antibody compared to the parental FR and CDR sequences (for instance compared to the omalizumab or omalizumab Fab sequences). As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

As used herein, the term “neutralizing” refers to neutralization of biological activity of IgE, when an anti-IgE antibody, or antigen binding agent, of the invention described herein specifically binds the IgE protein. Neutralizing may be the result of different ways of binding of said antibody to IgE. Preferably a neutralizing antibody is an antibody whose binding to IgE results in neutralization of a biological activity of IgE. Preferably the neutralizing binding protein binds IgE and decreases a biologically activity of IgE by at least about 5%, 10%, 15%, 20%, 25%, 30%, 35%, 40%, 50%, 60%, 80%, 85%, or more. Neutralization of a biological activity of IgE by a neutralizing antibody can be assessed by measuring one or more indicators of IgE biological activity described herein.

A “neutralizing monoclonal antibody” as used herein is intended to refer to a preparation of antibody molecules, which upon binding to IgE are able to inhibit or reduce the biological activity of IgE either partially or fully.

As used herein, the term “attenuation,” “attenuate,” and the like refers to the lessening or reduction in the severity of a symptom or condition caused by elevated serum IgE levels.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “kₒₙ”, as used herein, is intended to refer to the on rate constant for association of an antibody to the antigen to form the antibody/antigen complex as is known in the art.

The term “k_{off}”, as used herein, is intended to refer to the off rate constant for dissociation of an antibody from the antibody/antigen complex as is known in the art.

The term “k_{d}” or “k_{D}”, as used herein, is intended to refer to the dissociation constant of a particular antibody-antigen interaction as is known in the art.

The strength, or affinity of immunological binding interactions can be expressed in terms of the dissociation constant (k_{D} or k_{d}) of the interaction, wherein a smaller k_{d} represents a greater or higher affinity. Immunological binding properties of selected polypeptides can be quantified using methods well known in the art. One such method involves measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (“kₒₙ”) and the “off rate constant” (“K_{off}”) can be determined by calculation of the concentrations and the actual rates of association and dissociation. (Nature 361:186-87 (1993)). The ratio of k_{off}/kₒₙ enables the cancellation of all parameters not related to affinity, and is equal to the dissociation constant k_{d}. Davies et al. (1990) Annual Rev Biochem 59:439-473.

The term “antibody conjugate” refers to a binding protein, such as an antibody or antibody fragment or binding portion thereof, chemically linked to a second chemical moiety, such as a therapeutic or cytotoxic agent. The term “agent” is used herein to denote a chemical compound, a mixture of chemical compounds, a biological macromolecule, or an extract made from biological materials.

The terms “crystal”, and “crystallized” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N. Y., (1999).”

The term “polynucleotide” as referred to herein means a polymeric form of two or more nucleotides, either ribonucleotides or deoxynucleotides or a modified form of either type of nucleotide. The term includes single and double stranded forms of DNA but preferably is double-stranded DNA.

The term “isolated polynucleotide” as used herein means a polynucleotide (e.g., of genomic, cDNA, or synthetic origin, or some combination thereof) that, by virtue of its origin, the “isolated polynucleotide” is not associated with all or a portion of a polynucleotide with which the “isolated polynucleotide” is found in nature; is operably linked to a polynucleotide that it is not linked to in nature; or does not occur in nature as part of a larger sequence.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest.

The term “expression control sequence” as used herein refers to polynucleotide sequences, which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation,” as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g. prophylactic or therapeutic agent).

The specific region or epitope mapping of human IgE protein provided herein can be identified by any suitable epitope mapping method known in the art in combination with any one of the antibodies provided by the present invention. Examples of such methods include screening peptides of varying lengths derived from IgE for binding to the antibody of the present invention with the smallest fragment that can specifically bind to the antibody containing the sequence of the epitope recognized by the antibody. The IgE peptides may be produced synthetically or by proteolytic digestion of the IgE protein. Peptides that bind the antibody can be identified by, for example, mass spectrometric analysis. In another example, NMR spectroscopy or X-ray crystallography can be used to identify the epitope bound by an antibody of the present invention. Crystallization and X-ray crystallography techniques are preferred for determining the structure of IgE and the epitope on IgE that the anti-IgE antibody, or antigen binding agent, of the invention binds to.

Antibodies for use in the invention may be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA 93 (15): 7843-78481; WO92/02551; WO2004/051268 and International Patent Application number WO2004/106377. Screening for antibodies can be performed using assays to measure binding to human IgE and/or assays to measure the ability to block IgE binding to its natural receptor. An example of a binding assay is an ELISA.

Humanized antibodies (which include CDR-grafted antibodies) are antibody molecules having one or more complementarity determining regions (CDRs) from a non-human species (e.g., a rabbit or mouse) and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanized antibodies may optionally further comprise one or more framework residues derived from the non-human species from which the CDRs were derived. The latter are often referred to as donor residues. The antibody molecules of the present invention suitably have a binding affinity (K_{D}) of less than 2 nM. Affinity may be measured using any suitable method known in the art, including BIAcore, as described in the Examples herein (see Example 6), using isolated natural or recombinant IgE or a suitable fusion protein/polypeptide.

The affinity of an antibody or antigen binding agent of the present invention, as well as the extent to which a binding agent (such as an antibody) inhibits binding, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analysed to yield apparent rate constants for the association and dissociation phases of the binding reaction. The ratio of these values gives the apparent equilibrium constant (affinity) (see, e.g., Wolff et al, Cancer Res. 53:2560-65 (1993)).

It will be appreciated that the affinity of antibodies provided by the present invention may be altered using any suitable method known in the art. The present invention therefore also relates to variants of the antibody molecules of the present invention, which have an improved affinity for IgE. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

### Humanized Antibodies and Antigen Binding Agents

In one aspect of the present invention, provided herein are humanized anti-IgE monoclonal antibodies and antigen binding agents. Humanized antibodies are antibodies wherein the heavy and/or light chain contains one or more CDRs (including, if desired, one or more modified CDRs) from a donor antibody (e.g. a non-human antibody such as a murine or rabbit monoclonal antibody) grafted into a heavy and/or light chain variable region framework of an acceptor antibody (e.g. a human antibody). For a review, see Vaughan et al, Nature Biotechnology, 16, 535-539, 1998.

In one embodiment, rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein are transferred to the human antibody framework. In another embodiment only the specificity determining residues from each of the CDRs described herein are transferred to the human antibody framework.

When the CDRs or specificity determining residues are grafted, any appropriate acceptor variable region framework sequence may be used having regard to the class/type of the donor antibody from which the CDRs are derived, including mouse, rabbit, primate and human framework regions.

Suitably, the humanized antibody according to the present invention has a variable domain comprising human acceptor framework regions as well as one or more of the CDRs provided specifically herein. Thus, provided in one embodiment is a humanized monoclonal antibody which binds human IgE wherein the variable domain comprises human acceptor framework regions (with optional mutations as described herein) and non-human donor CDRs.

Construction of CDR-grafted antibodies is generally described in European Patent Application EP-A-0239400, which discloses a process in which the CDRs of a mouse monoclonal antibody are grafted onto the framework regions of the variable domains of a human immunoglobulin by site directed mutagenesis using long oligonucleotides, and is incorporated herein. The CDRs determine the antigen binding specificity of antibodies and are relatively short peptide sequences carried on the framework regions of the variable domains.

The earliest work on humanizing monoclonal antibodies by CDR-grafting was carried out on monoclonal antibodies recognizing synthetic antigens, such as NP. However, examples in which a mouse monoclonal antibody recognizing lysozyme and a rat monoclonal antibody recognizing an antigen on human T-cells were humanized by CDR-grafting have been described by Verhoeyen et al. (Science, 239, 1534-1536, 1988) and Riechmann et al (Nature, 332, 323-324, 1988), respectively. Antibody humanization is achieved by grafting CDRs of a non-human antibody, such as a mouse, rat, goat, or rabbit antibody, onto a “similar” human framework (acceptor) and selecting minimal number of key framework residues (back-mutations) that are manually selected from the donor monoclonal antibody and incorporated into human acceptor framework in order to maintain the original CDR conformation. Such methods are known in the art, and include those described in Jones et al., Nature 321:522 (1986); Verhoeyen et al., Science 239:1534 (1988)), Sims et al., J. Immunol. 151:2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28 (4/5): 489-498 (1991); Studnicka et al., Protein Engineering 7 (6): 805-814 (1994); Roguska. et al., PNAS 91:969-973 (1994); PCT publication WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, which are incorporated herein.

The human variable heavy and light chain germline subfamily classification can be derived from the Kabat germline subgroup designations: VH1, VH2, VH3, VH4, VH5, VH6 or VH7 for a particular VH sequence and JH1, JH2, JH3, JH4, JH5, and JH6 for a for a particular variable heavy joining group for framework 4; VK1, VK2, VK3, VK4, VK5 or VK6 for a particular VL kappa sequence for framework 1, 2, and 3, and JK1, JK2, JK3, JK4, or JK5 for a particular kappa joining group for framework 4; or VL1, VL2, VL3, VL4, VL5, VL6, VL7, VL8, VL9, or VL10 for a particular VL lambda sequence for framework 1, 2, and 3, and JL1, JL2, JL3, or JL 7 for a particular lambda joining group for framework 4.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular embodiments, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example IgG4 molecules in which the serine at position 241 has been changed to proline as described in Angal et al., Molecular Immunology, 1993, 30 (1), 105-108 may be used. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

### CDR and Human Framework Modifications

Riechmann et al., found that the transfer of the CDRs alone (as defined by Kabat (Kabat et al. (supra) and Wu et al., J. Exp. Med., 132, 211-250, 1970)) was not sufficient to provide satisfactory antigen binding activity in the CDR-grafted product. It was found that a number of framework residues have to be altered so that they correspond to those of the donor framework region. Proposed criteria for selecting which framework residues need to be altered are described in International Patent Application WO 90/07861, which is incorporated herein.

The substitution of non-human CDRs into a human variable domain framework is most likely to result in retention of the CDR's correct spatial orientation if the human variable domain framework adopts the same or similar conformation to the non-human variable framework from which the CDRs originated. This is achieved by obtaining the human variable domains from human antibodies whose framework sequences exhibit a high degree of sequence identity with the non-human variable framework domains from which the CDRs were derived. As described above, the heavy and light chain variable framework regions can be derived from the same or different human antibody sequences. The human antibody sequences can be the sequences of naturally occurring human antibodies or can be consensus sequences of several human antibodies. See Kettleborough et al, Protein Engineering 4:773 (1991); Kolbinger et al., Protein Engineering 6:971 (1993) and Carter et al, WO 92/22653.

Having identified the complementarity determining regions of the non-human donor immunoglobulin and appropriate human acceptor immunoglobulins, the next step is to determine which, if any, residues from these components should be substituted to optimize the properties of the resulting humanized antibody. In general, substitution of human amino acid residues with non-human amino acid residues should be minimized, because introduction of non-human residues increases the risk of the antibody eliciting a human-anti-donor-antibody (HADA) response in humans. Art-recognized methods of determining immune response can be performed to monitor a HADA response in a particular host or during clinical trials. Hosts administered humanized antibodies can be given an immunogenicity assessment at the beginning and throughout the administration of said therapy. The HADA response is measured, for example, by detecting antibodies to the humanized therapeutic reagent, in serum samples from the host using a method known to one in the art, including surface plasmon resonance technology (BIACORE) and/or solid-phase ELISA analysis.

The selection of amino acid residues for substitution (also “mutation” herein) is determined, in part, by computer modelling. Computer hardware and software are described herein for producing three-dimensional images of immunoglobulin molecules. In general, molecular models are produced starting from solved structures for immunoglobulin chains or domains thereof. The chains to be modelled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modelling, and preferably those sharing at least 60%, 70%, 80%, 90%, sequence identity or more are selected for modelling. The solved starting structures are modified to allow for differences between the actual amino acids in the immunoglobulin chains or domains being modelled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

The selection of amino acid residues for substitution can also be determined, in part, by examination of the characteristics of the amino acids at particular locations, or empirical observation of the effects of substitution or mutagenesis of particular amino acids. For example, when an amino acid differs between a donor variable region framework residue and a selected human variable region framework residue, the human framework amino acid should usually be substituted by the equivalent framework amino acid from the donor antibody when it is reasonably expected that the amino acid:
(1) noncovalently binds antigen directly,
(2) is adjacent to a CDR region,
(3) otherwise interacts with a CDR region (e.g., is within about 3-6 Angstrom of a CDR region as determined by computer modelling), or
(4) participates in the VL-VH interface.

Residues which “noncovalently bind antigen directly” include amino acids in positions in framework regions which have a good probability of directly ‘interacting with amino acids on the antigen according to established chemical forces, for example, by hydrogen bonding, Van der Waals forces, hydrophobic interactions, and the like. CDR and framework regions are as defined by Kabat et al. or Chothia et al, supra. When framework residues, as defined by Kabat et al, supra, constitute structural loop residues as defined by Chothia et al, supra, the amino acids present in the donor antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk 1 MB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al, Science, 233:747 (1986), which is incorporated herein by reference) and selecting these amino acids from the donor may be desirable to keep all the antigen contacts that provide affinity in the original antibody. As described herein, FR sequences may also be substituted/mutated to improve the affinity of the anti-IgE antibody, or antigen binding agent, of the invention for IgE (and/or to extend its interaction or epitope on Cε2 of IgE).

Residues that “otherwise interact with a CDR region” include those that are determined by secondary structural analysis to be in a spatial orientation sufficient to effect a CDR region. In one embodiment, residues that “otherwise interact with a CDR region” are identified by analysing a three-dimensional model of the donor immunoglobulin (e.g., a computer-generated model). A three-dimensional model, typically of the original donor antibody, shows that certain amino acids outside of the CDRs are close to the CDRs and have a good probability of interacting with amino acids in the CDRs by hydrogen bonding, Van der Waals forces, hydrophobic interactions, etc. At those amino acid positions, the donor immunoglobulin amino acid rather than the acceptor immunoglobulin amino acid may be selected. Amino acids according to this criterion will generally have a side chain atom within about 3 angstrom units (A) of some atom in the CDRs and must contain an atom that could interact with the CDR atoms according to established chemical forces, such as those listed above. In the case of atoms that may form a hydrogen bond, the 3 A° is measured between their nuclei, but for atoms that do not form a bond, the 3 A° is measured between their Van der Waals surfaces. Hence, in the latter case, the nuclei must be within about 6 A° (3 A plus the sum of the Van der Waals radii) for the atoms to be considered capable of interacting. In many cases the nuclei will be from 4 or 5 to 6 A° apart. In determining whether an amino acid can interact with the CDRs, it is preferred not to consider the last 8 amino acids of heavy chain CDR 2 as part of the CDRs, because from the viewpoint of structure, these 8 amino acids behave more as part of the framework.

Amino acids that are capable of interacting with amino acids in the CDRs (or FRs), may be identified in yet another way. The solvent accessible surface area of each framework amino acid is calculated in two ways: (1) in the intact antibody, and (2) in a hypothetical molecule consisting of the antibody with its CDRs removed. A significant difference between these numbers of about 10 square angstroms or more shows that access of the framework amino acid to solvent is at least partly blocked by the CDRs, and therefore that the amino acid is making contact with the CDRs. Solvent accessible surface area of an amino acid may be calculated based on a three-dimensional model of an antibody, using algorithms known in the art (e.g., Connolly, J. Appl. Cryst. 16:548 (1983) and Lee and Richards, J. Mol. Biol. 55:379 (1971), both of which are incorporated herein by reference). Framework amino acids may also occasionally interact with the CDRs indirectly, by affecting the conformation of another framework amino acid that in turn contacts the CDRs.

Particular amino acids at several positions in the framework are known to be capable of interacting with the CDRs in many antibodies (Chothia and Lesk, supra, Chothia et al, supra and Tramontano et al, J. Mol. Biol. 215:175 (1990), all of which are incorporated herein by reference). Notably, the amino acids at positions 2, 48, 64, and 71 of the light chain and 71 and 94 of the heavy chain (numbering according to Kabat) are known to be capable of interacting with the CDRs in many antibodies. The amino acids at positions 35 in the light chain and 93 and 103 in the heavy chain are also likely to interact with the CDRs. At all these numbered positions, choice of the donor amino acid rather than the acceptor amino acid (when they differ) to be in the humanized immunoglobulin is preferred. On the other hand, certain residues capable of interacting with the CDR region, such as the first 5 amino acids of the light chain, may sometimes be chosen from the acceptor immunoglobulin without loss of affinity in the humanized immunoglobulin.

Residues which “participate in the VL-VH interface” or “packing residues” include those residues at the interface between VL and VH as defined, for example, by Novotny and Haber, Proc. Natl. Acad. Sci. USA, 82:4592-66 (1985) or Chothia et al, supra. Generally, unusual packing residues should be retained in the humanized antibody if they differ from those in the human frameworks.

In general, one or more of the amino acids fulfilling the above criteria is substituted. In some embodiments, all or most of the amino acids fulfilling the above criteria are substituted. Occasionally, there is some ambiguity about whether a particular amino acid meets the above criteria, and alternative variant immunoglobulins are produced, one of which has that particular substitution, the other of which does not. Alternative variant immunoglobulins so produced can be tested in any of the assays described herein for the desired activity, and the preferred immunoglobulin selected.

Usually the CDR regions in humanized antibodies are substantially identical, and more usually, identical to the corresponding CDR regions of the donor antibody. Although not usually desirable, it is sometimes possible to make one or more conservative amino acid substitutions of CDR residues without appreciably affecting the binding affinity of the resulting humanized immunoglobulin. By conservative or similar substitutions is intended combinations such as, for example, leucine being substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and,
cysteine and methionine (amino acids having sulphur-containing side chains).

Additional candidates for substitution are acceptor human framework amino acids that are unusual or “rare” for a human immunoglobulin at that position. These amino acids can be substituted with amino acids from the equivalent position of the donor antibody or from the equivalent positions of more typical human immunoglobulins. For example, substitution may be desirable when the amino acid in a human framework region of the acceptor immunoglobulin is rare for that position and the corresponding amino acid in the donor immunoglobulin is common for that position in human immunoglobulin sequences; or when the amino acid in the acceptor immunoglobulin is rare for that position and the corresponding amino acid in the donor immunoglobulin is also rare, relative to other human sequences. These criteria help ensure that an atypical amino acid in the human framework does not disrupt the antibody structure. Moreover, by replacing an unusual human acceptor amino acid with an amino acid from the donor antibody that happens to be typical for human antibodies, the humanized antibody may be made less immunogenic.

The term “rare”, as used herein, indicates an amino acid occurring at that position in less than about 20% but usually less than about 10% of sequences in a representative sample of sequences, and the term “common,” as used herein, indicates an amino acid occurring in more than about 25% but usually more than about 50% of sequences in a representative sample. For example, all human light and heavy chain variable region sequences are respectively grouped into “subgroups” of sequences that are especially homologous to each other and have the same amino acids at certain critical positions (Kabat et al, supra). When deciding whether an amino acid in a human acceptor sequence is “rare” or “common” among human sequences, it will often be preferable to consider only those human sequences in the same subgroup as the acceptor sequence.

Additional candidates for substitution are acceptor framework residues that correspond to a rare or unusual donor framework residue. Rare or unusual donor framework residues are those that are rare or unusual (as defined herein) for donor antibodies at that position. For donor antibodies, the subgroup can be determined according to Kabat and residue positions identified which differ from the consensus. These donor specific differences may point to somatic mutations in the donor sequence, which enhance activity. Unusual residues that are predicted to affect binding are retained, whereas residues predicted to be unimportant for binding could be substituted.

Additional candidates for substitution are non-germline residues occurring in an acceptor framework region. For example, when an acceptor antibody chain (i.e., a human antibody chain sharing significant sequence identity with the donor antibody chain) is aligned to a germline antibody chain (likewise sharing significant sequence identity with the donor chain), residues not matching between acceptor chain framework and the germline chain framework can be substituted with corresponding residues from the germline sequence.

Other than the specific amino acid substitutions discussed above, the framework regions of humanized immunoglobulins are usually substantially identical, and more usually, identical to the framework regions of the human antibodies from which they were derived (except as described herein for the purposes of the present invention). Of course, many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin. Thus, in one embodiment the variable framework region of the humanized immunoglobulin shares at least 65, 75 or 85% sequence similarity or identity to a human variable framework region sequence or consensus of such sequences. In another embodiment, the variable framework region of the humanized immunoglobulin shares at least 90%, preferably 95%, more preferably 96%, 97%, 98%, or 99%, sequence similarity or identity to a human variable framework region sequence or consensus of such sequences. In general, however, such substitutions are undesirable (except those described herein).

As used herein, degrees of identity and similarity can be readily calculated, for example as described in Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656, which are incorporated by reference herein.

A number of reviews discussing CDR-grafted antibodies have been published, including Vaughan et al. (Nature Biotechnology, 16, 535-539, 1998), which is incorporated by reference herein.

The anti-IgE antibodies of the present invention may include further additional binding domains for example as per the molecule DVD-Ig as disclosed in WO 2007/024715, or the so-called (FabFv) 2Fc described in WO2011/030107. Thus, antibody as employed herein includes bi, tri or tetra-valent full length antibodies.

### Antigen Binding Agents

Antigen binding agents include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH) for example as described in WO 2001090190, scFv, bi, tri or tetra-valent antibodies, Bis-scFv, diabodies, tribodies, triabodies, tetrabodies and epitope-antigen binding agents of any of the above (see for example Holliger and Hudson, 2005, Nature Biotech. 23 (9): 1126-1136; Adair and Lawson, 2005, Drug Design Reviews-Online 2 (3), 209-217). The methods for creating and manufacturing these antibody fragments are well known in the art (see for example Verma et al., 1998, Journal of Immunological Methods, 216, 165-181). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170, and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583.

A typical Fab′ molecule comprises a heavy and a light chain pair in which the heavy chain comprises a variable region VH, a constant domain CH1 and a natural or modified hinge region and the light chain comprises a variable region VL and a constant domain CL.

In one embodiment, there is provided a dimer of a Fab′ according to the present disclosure to create a F (ab′)2 for example dimerization may be through a natural hinge sequence described herein, or derivative thereof, or a synthetic hinge sequence.

An antibody binding domain will generally comprise 6 CDRs, three from a heavy chain and three from a light chain. In one embodiment, the CDRs are in a framework and together form a variable region. Thus in one embodiment, the antigen binding agent includes a binding domain specific for IgE comprising a light chain variable region and a heavy chain variable region.

It will be appreciated that one or more (for example 1, 2, 3 or 4) amino acid substitutions, additions and/or deletions may be made to the CDRs or other sequences (e.g variable domains) provided by the present invention, as described above or below, without significantly altering the ability of the antibody to bind to IgE. The effect of any amino acid substitutions, additions and/or deletions can be readily tested by one skilled in the art, for example by using the methods described herein, in particular in the Examples.

In one embodiment, one or more (for example 1, 2, 3 or 4) amino acid substitutions, additions and/or deletions may be made to the CDRs or framework region employed in the antibody or fragment provided by the present invention so that the binding affinity (K_{D}) of the anti-IgE antibody, or antigen binding agent, of the invention to IgE is less than 2, 1.9, 1.8, 1.7, 1.6, 1.5, 1.4, 1.3, 1.2, 1.1, 1.0, 0.9, 0.8, 0.7, 0.6, 0.5, 0.4, or 0.3 nM. In one embodiment, provided is a modified humanized antibody wherein modifications have been made to either the CDRs, framework regions, or both, in order to decrease the Kn for example to less than 2, 1.9, 1.8, 1.7, 1.6, 1.5, 1.4, 1.3, 1.2, 1.1, 1.0, 0.9, 0.8, 0.7, 0.6, 0.5, 0.4, or 0.3 nM.

The antibody fragment of the present invention includes Fab, Fab′, F(ab′)2, scFv, diabody, scFAb, dFv, single domain light chain antibodies, dsFv, a peptide comprising CDR, and the like.

A Fab is an antibody fragment having a molecular weight of about 50,000 and antigen binding activity, in which about a half of the N-terminal side of H chain and the entire L chain, among fragments obtained by treating IgG with a protease, papain (cut at an amino acid residue at position 224 of the H chain), are bound together through a disulfide bond.

The Fab of the present invention can be obtained by treating a humanised CDR-grafted antibody of the present invention which specifically reacts with IgE, with a protease, papain. Also, the Fab can be produced by inserting DNA encoding Fab of the antibody into an expression vector for prokaryote or an expression vector for eukaryote, and introducing the vector into a prokaryote or eukaryote to express the Fab.

An F (ab′)2 is an antibody fragment having a molecular weight of about 100,000 and antigen binding activity, which is slightly larger than the Fab bound via a disulfide bond of the hinge region, among fragments obtained by treating IgG with a protease, pepsin.

The F (ab′)2 of the present invention can be obtained by treating a human CDR-grafted antibody which specifically reacts with IgE, with a protease, pepsin. Also, the F (ab′)2 can be produced by binding Fab′ described below via a thioether bond or a disulfide bond.

A Fab′ is an antibody fragment having a molecular weight of about 50,000 and antigen binding activity, which is obtained by cutting a disulfide bond of the hinge region of the F (ab′)2.

The Fab′ of the present invention can be obtained by treating the F (ab′)2 which specifically reacts with IgE, with a reducing agent, dithiothreitol. Also, the Fab′ of the present invention can be produced by inserting DNA encoding a Fab′ of a human CDR-grafted antibody of the present invention which specifically reacts with IgE into an expression vector for prokaryote or an expression vector for eukaryote, and introducing the vector into a prokaryote or eukaryote to express the Fab′.

An scFv is a VH-P-VL or VL-P-VH polypeptide in which one chain VH and one chain VL are linked using an appropriate peptide linker (P) of 12 or more residues and which has an antigen-binding activity.

The scFv of the present invention can be produced by obtaining cDNAs encoding VH and VL of a human CDR-grafted antibody which specifically reacts with IgE of the present invention, constructing DNA encoding scFv, inserting the DNA into an expression vector for prokaryote or an expression vector for eukaryote, and then introducing the expression vector into a prokaryote or eukaryote to express the scFv.

The Fab fragment of the present invention may be linked, directly or via a linker to a scFv. “Single chain variable fragment” or “scFv” as employed herein refers to a single chain variable fragment which is stabilized by a peptide linker between the VH and VL variable domains, for example a peptide linker with amino acid sequence which is Seq ID No. 151. The linkage to the Fab fragment can be a chemical conjugation but is most preferably a translation fusion, i.e. a genetic fusion where the sequence of each is encoded in sequence by an expression vector. The linker is therefore typically an amino acid linker as described herein. The scFv of the present invention linked to the Fab fragment may bind to a serum carrier protein in order to extend the half-life of the antibody fusion protein in vivo. Extending half-life in such a way is independent of IgE binding and may be advantageous.

“Serum carrier protein” as employed herein refers to any suitable plasma carrier protein to which the scFv may bind, in one example the serum carrier protein is selected from thyroxine binding protein, transthyretin, al-acid glycoprotein, transferrin, fibrinogen and albumin, or a fragment of any thereof. Typically, the scFv binds to albumin, preferably human serum albumin.

Any suitable albumin binding scFv may be incorporated into the antibody fusion proteins of the invention. Suitable albumin binding domains have previously been described in the art.

A diabody is an antibody fragment in which scFv's having the same or different antigen binding specificity forms a dimer, and has a divalent antigen binding activity to the same antigen or two specific antigen binding activities to different antigens.

The diabody of the present invention, for example, a divalent diabody which specifically reacts with IgE, can be produced by obtaining cDNAs encoding VH and VL of an antibody which specifically reacts with IgE, constructing DNA encoding scFv having a polypeptide linker of 3 to 10 residues, inserting the DNA into an expression vector for prokaryote or an expression vector for eukaryote, and then introducing the expression vector into a prokaryote or eukaryote to express the diabody.

A dsFv is obtained by binding polypeptides in which one amino acid residue of each of VH and VL is substituted with a cysteine residue via a disulfide bond between the cysteine residues. The amino acid residue, which is substituted with a cysteine residue can be selected based on a three-dimensional structure estimation of the antibody in accordance with the method shown by Reiter et al. (Protein Engineering, 7, 697 (1994)).

The dsFv of the present invention can be produced by obtaining cDNAs encoding VH and VL of a human CDR-grafted antibody which specifically reacts with IgE of the present invention, constructing DNA encoding dsFv, inserting the DNA into an expression vector for prokaryote or an expression vector for eukaryote, and then introducing the expression vector into a prokaryote or eukaryote to express the dsFv.

A peptide comprising CDR is constituted by including at least one region of H chain and L chain CDRs. Plural CDRs can be bound directly or via an appropriate peptide linker.

The peptide comprising CDR of the present invention can be produced by obtaining cDNA encoding CDR of VH and VL of a human CDR-grafted antibody which specifically reacts with IgE, constructing DNA encoding CDR, inserting the DNA into an expression vector for prokaryote or an expression vector for eukaryote, and then by introducing the expression vector into a prokaryote or eukaryote to express the peptide. Also, the peptide comprising CDR can also be produced by a chemical synthesis method such as an Fmoc method (fluorenylmethoxycarbonyl method), a tBoc method (t-butyloxycarbonyl method), or the like.

The antibody of the present invention includes antibody derivatives in which a radioisotope, a protein, an agent or the like is chemically or genetically conjugated to the antibody of the present invention.

The antibody derivatives of the present invention can be produced by chemically conjugating a radioisotope, a protein or an agent to the N-terminal side or C-terminal side of an H chain or an L chain of an antibody or antibody fragment which specifically reacts with IgE, to an appropriate substituent group or side chain of the antibody or antibody fragment or to a sugar chain in the antibody or antibody fragment (Antibody Engineering Handbook, edited by Osamu Kanemitsu, published by Chijin Shokan (1994)).

Also, it can be genetically produced by linking a DNA encoding the antibody or the antibody fragment of the present invention which specifically reacts with IgE to other DNA encoding a protein to be bound, inserting the DNA into an expression vector, and introducing the expression vector into a host cell.

The radioisotope includes 1311, 1251 and the like, and it can be conjugated to the antibody by, e.g., a chloramine T method.

The agent is preferably a low molecular weight compound. Examples include anticancer agents such as alkylating agents (e.g., nitrogen mustard, cyclophosphamide), metabolic antagonists (e.g., 5-fluorouracil, methotrexate), antibiotics (e.g., daunomycin, bleomycin, mitomycin C, daunorubicin, doxorubicin), plant alkaloids (e.g., vincristine, vinblastine, vindesine), hormone drugs (e.g., tamoxifen, dexamethasone), and the like (Clinical Oncology, edited by Japanese Society of Clinical Oncology, published by Cancer and Chemotherapy (1996)); anti-inflammatory agents such as steroid agents (e.g., hydrocortisone, prednisone), non-steroidal drugs (e.g., aspirin, indometacin), immunomodulators (e.g., aurothiomalate, penicillamine), immunosuppressing agents (e.g., cyclophosphamide, azathioprine) and antihistaminic agents (e.g., chlorpheniramine maleate, clemastine) (Inflammation and Anti-inflammatory Therapy, Ishiyaku Shuppan (1982)); and the like. The method for conjugating daunomycin to an antibody includes a method in which daunomycin and an amino group of an antibody are conjugated via glutaraldehyde, a method in which an amino group of daunomycin and a carboxyl group of an antibody are conjugated via a water-soluble carbodiimide, and the like.

Also, in order to inhibit cancer cells directly, a toxin such as ricin, diphtheria toxin and the like, can be used. For example, a fusion antibody with a protein can be produced by linking a cDNA encoding an antibody or antibody fragment to other cDNA encoding the protein, constructing DNA encoding the fusion antibody, inserting the DNA into an expression vector for prokaryote or an expression vector for eukaryote, and then introducing it into a prokaryote or eukaryote to express the fusion antibody.

Further contemplated herein are antibody fragments or antigen binding agents including fusions of binding agents, for example immunoglobulin like fragments and agents such as diabodies, scAbs, bispecific fragments, triabodies, Fab-Fv-Fv, Fab-Fv, tribody, (Fab-Fv) 2-Fc, and antibody fragments or portions, such as CDRs or antibody loops including CDRs, which are grafted onto non-Ig frameworks such as fibronectin or leucine zippers, as descried in Binz et al., (2005) Nat. Biotech. 23:1257-1268, incorporated in its entirety herein.

### Conjugated Anti-IgE Monoclonal Antibodies and Antigen Binding Agents

If desired, an antibody or antigen binding agent for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, antigen binding agents, synthetic (including PEG) or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as 111 In and 90Y, Lu177, Bismuth213, Californium252, Iridium 192 and Tungsten188/Rhenium188; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, a-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111 In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

In one embodiment a half-life provided by an effector molecule which is independent of IgE or an anti-human IgE antibody is advantageous.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo—or hetero-polysaccharide.

Specific optional substituents, which may be present on the above-mentioned synthetic polymers, include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof. In one embodiment the polymer is a PEG molecule.

“Derivatives” as used herein in regard to conjugates is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the natural or synthetic polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above.

The activated polymer may be any polymer containing a thiol reactive group such as an «-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido) propionamido]ethyl amide of N,N′-bis(methoxypoly (ethylene glycol) MW 20,000) modified lysine, also known as PEG2MAL40K (obtainable from Nektar, formerly Shearwater).

Alternative sources of PEG linkers include NOF who supply GL2-400MA3 (wherein m in the structure below is 5) and GL2-400MA (where m is 2) and n is approximately 450:

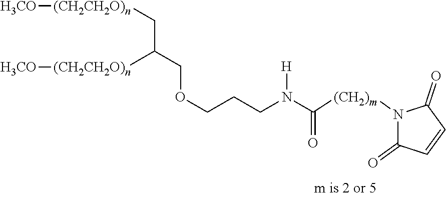

That is to say each PEG is about 20,000 Da.

Thus in one embodiment the PEG is 2,3-Bis(methylpolyoxyethylene-oxy)-1-{[3-(6-maleimido-1-oxohexyl) amino]propyloxy}hexane (the 2 arm branched PEG, —CH2) 3NHCO (CH2) 5-MAL, Mw 40,000 known as SUNBRIGHT GL2-400MA3.

Further alternative PEG effector molecules of the following type:

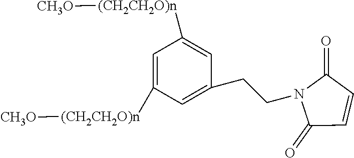

are available from Dr Reddy, NOF and Jenkem.

In one embodiment there is provided an antibody of the invention which is PEGylated (for example with a PEG described herein), attached through a cysteine amino acid residue at or about amino acid 226 in the chain, for example amino acid 226 of the heavy chain (by sequential numbering).

In one embodiment the present disclosure provides a Fab′PEG molecule comprising one or more PEG polymers, for example 1 or 2 polymers such as a 40 kDa polymer or polymers.

Fab′-PEG molecules according to the present disclosure may be particularly advantageous in that they have a half-life independent of the Fc fragment. In one example the present invention provides a method treating a disease ameliorated by modulating human IgE biological activity comprising administering a therapeutically effective amount of an anti-IgE antibody or antigen binding agent thereof wherein the antibody or antigen binding agent thereof has a half-life that is independent of Fc binding to IgE.

In one embodiment there is provided a Fab′ conjugated to a polymer, such as a PEG molecule, a starch molecule or an albumin molecule.

In one embodiment there is provided a scFv conjugated to a polymer, such as a PEG molecule, a starch molecule or an albumin molecule.

In one embodiment the antibody or fragment is conjugated to a starch molecule, for example to increase the half-life. Methods of conjugating starch to a protein as described in U.S. Pat. No. 8,017,739 incorporated herein by reference.

### Polynucleotides

The present invention also provides an isolated DNA sequence encoding the heavy and/or light chain(s) of an antibody molecule of the present invention. Suitably, the DNA sequence encodes the heavy or the light chain of an antibody molecule of the present invention. The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

DNA coding for acceptor framework sequences is widely available to those skilled in the art and can be readily synthesisedsynthesized on the basis of their known amino acid sequences.

Standard techniques of molecular biology may be used to prepare DNA sequences coding for the antibody molecule of the present invention. Desired DNA sequences may be synthesized completely or in part using oligonucleotide synthesis techniques. Site-directed mutagenesis and polymerase chain reaction (PCR) techniques may be used as appropriate.

The present invention also relates to a cloning or expression vector comprising one or more DNA sequences of the present invention. Accordingly, provided is a cloning or expression vector comprising one or more DNA sequences encoding an antibody of the present invention. Suitably, the cloning or expression vector comprises two DNA sequences, encoding the light chain and the heavy chain of the antibody molecule of the present invention, respectively and suitable signal sequences. In one example the vector comprises an intergenic sequence between the heavy and the light chains (see WO03/048208).

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbour Publishing.

Host Cells Expressing anti-IgE Antibodies or Fragments Thereof

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

Suitable types of Chinese Hamster Ovary (CHO cells) for use in the present invention may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NSO myeloma cells and SP2 cells, COS cells. Other suitable cells may include human embryonic kidney (hek) fibroblasts, for example hek293F and ExpiHek cells, which are known in the art.

CHO is preferred for full-length Ab of the invention, given this is standard host for the production of omalizumab (in one embodiment giving the antibodies of the invention the standard glycosylation pattern of omalizumab) [see also WO 2013/181577].

Production of anti-IgE Antibodies or Fragments Thereof

The present invention also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

There is a provided a process for culturing a host cell and expressing an antibody or fragment thereof, isolating the latter and optionally purifying the same to provide an isolated antibody or fragment. In one embodiment the process further comprises the step of conjugating an effector molecule to the isolated antibody or fragment, for example conjugating to a PEG polymer in particular as described herein.

In one embodiment there is provided a process for purifying an antibody (in particular an antibody or fragment according to the invention) comprising the steps: performing anion exchange chromatography in non-binding mode such that the impurities are retained on the column and the antibody is eluted.

In one embodiment the purification employs affinity capture on a Protein A column, and then titration. On one embodiment, the purification employs affinity capture on a Protein G column, and then HPLC titration. On one embodiment, the purification employs affinity capture on an IgE column, and then titration.

In one embodiment the purification employs cibacron blue or similar for purification of albumin fusion or conjugate molecules.

Suitable ion exchange resins for use in the process include Q.FF resin (supplied by GE-Healthcare). The step may, for example be performed at a pH about 8.

The process may further comprise an initial capture step employing cation exchange chromatography, performed for example at a pH of about 4 to 5, such as 4.5. The cation exchange chromatography may, for example employ a resin such as CaptoS resin or SP sepharose FF (supplied by GE-Healthcare). The antibody or fragment can then be eluted from the resin employing an ionic salt solution such as sodium chloride, for example at a concentration of 200 mM.

Thus the chromatograph step or steps may include one or more washing steps, as appropriate.

The purification process may also comprise one or more filtration steps, such as a diafiltration step or HPLC filtration step.

Thus in one embodiment there is provided a purified anti-IgE antibody or fragment, for example a humanised antibody or fragment, in particular an antibody or fragment according to the invention, in substantially purified from, in particular free or substantially free of endotoxin and/or host cell protein or DNA.

Purified from as used supra is intended to refer to at least 90% purity, such as 91, 92, 93, 94, 95, 96, 97, 98, 99% w/w or purer.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

### Pharmaceutical Compositions

As the antibodies of the present invention are useful in the treatment and/or prophylaxis of a pathological condition, the present invention also provides a pharmaceutical or diagnostic composition comprising an antibody or antigen binding agent of the present invention in combination with one or more of a pharmaceutically acceptable excipient, diluent, or carrier. Accordingly, provided is the use of an antibody or antigen binding agent of the invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The present invention also provides a process for preparation of a pharmaceutical or diagnostic composition comprising adding and mixing the antibody or antigen binding agent of the present invention together with one or more of a pharmaceutically acceptable excipient, diluent, or carrier.

The antibody or antigen binding agent may be the sole active ingredient in the pharmaceutical or diagnostic composition or may be accompanied by other active ingredients including other antibody ingredients or non-antibody ingredients such as steroids or other drug molecules, in particular drug molecules whose half-life is independent of IgE binding.

The pharmaceutical compositions suitably comprise a therapeutically effective amount of the antibody or antigen binding agent of the invention. The term “therapeutically effective amount” as used herein refers to an amount of a therapeutic agent needed to treat, ameliorate, or prevent a targeted disease or condition, or to exhibit a detectable therapeutic or preventative effect. For any disclosed antibody or antigen binding agent, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 500 mg/kg, for example 0.1 mg/kg to 200 mg/kg, such as 100 mg/Kg.

Pharmaceutical compositions may be conveniently presented in unit dose forms containing a predetermined amount of an active agent of the invention per dose.

Therapeutic doses of the antibodies or antigen binding agents according to the present disclosure show no apparent toxicology effects in vivo.

Advantageously, the levels of IgE activity in vivo may be maintained at an appropriately reduced level by administration of sequential doses of the antibody or binding agent according to the disclosure.

Compositions may be administered individually to a patient or may be administered in combination (e.g. simultaneously, sequentially, or separately) with other agents, drugs or hormones.

A pharmaceutical composition may also contain a pharmaceutically acceptable carrier for administration of the antibody or antigen binding agent. The carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Preferred forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, it is preferred that the compositions are adapted for administration to human subjects.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously (in particular), intraperitoneally, intravenously or intramuscularly, or delivered to the interstitial space of a tissue. The compositions can also be administered into a lesion. Dosage treatment may be a single dose schedule or a multiple dose schedule.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

Structural-functional properties

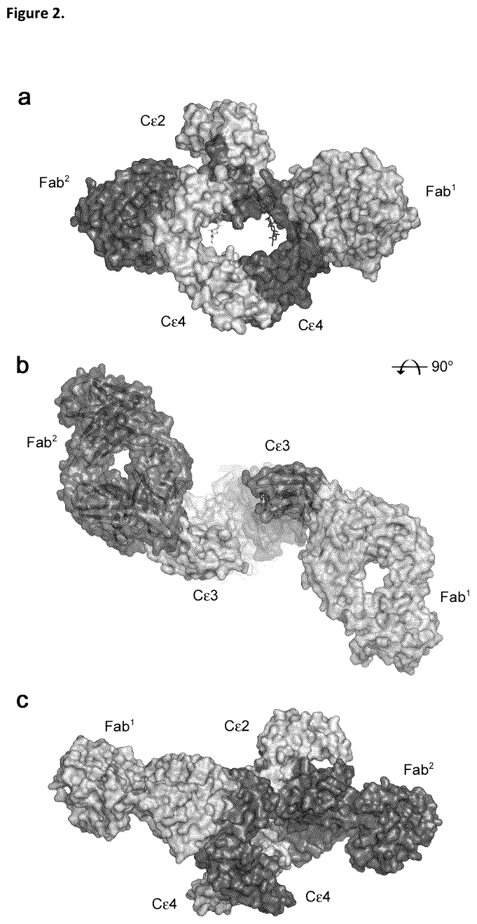

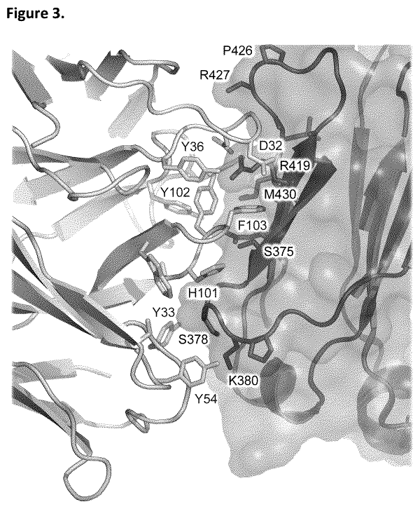

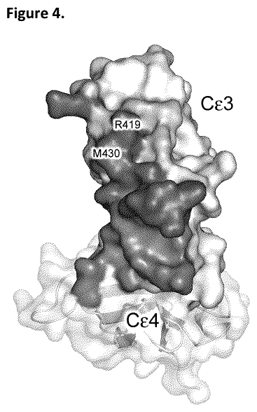

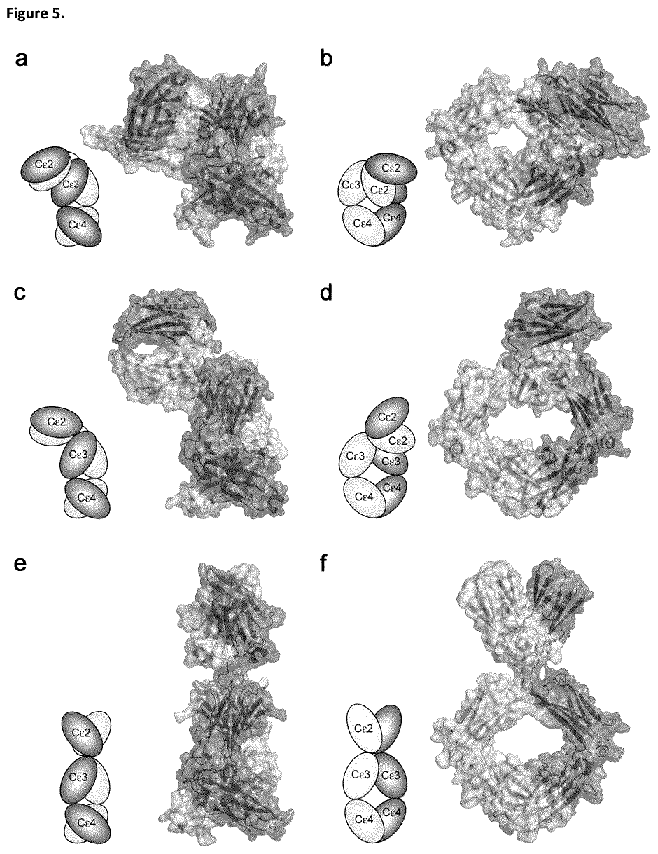

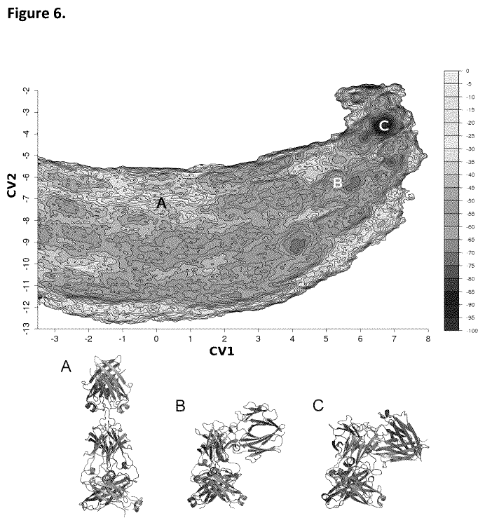

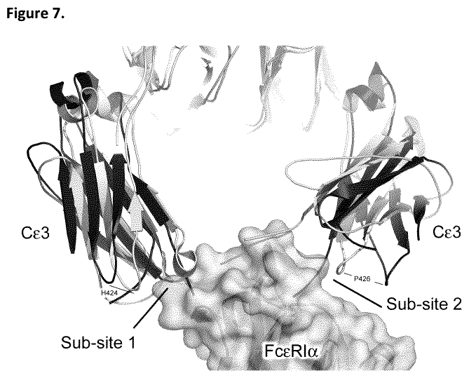

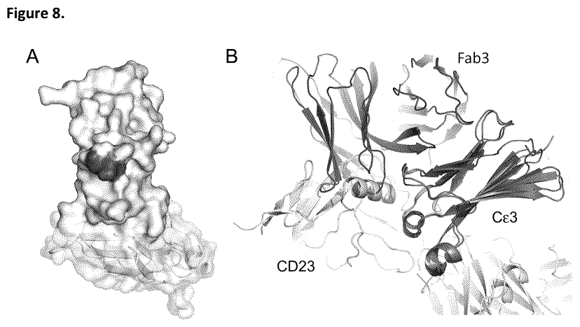

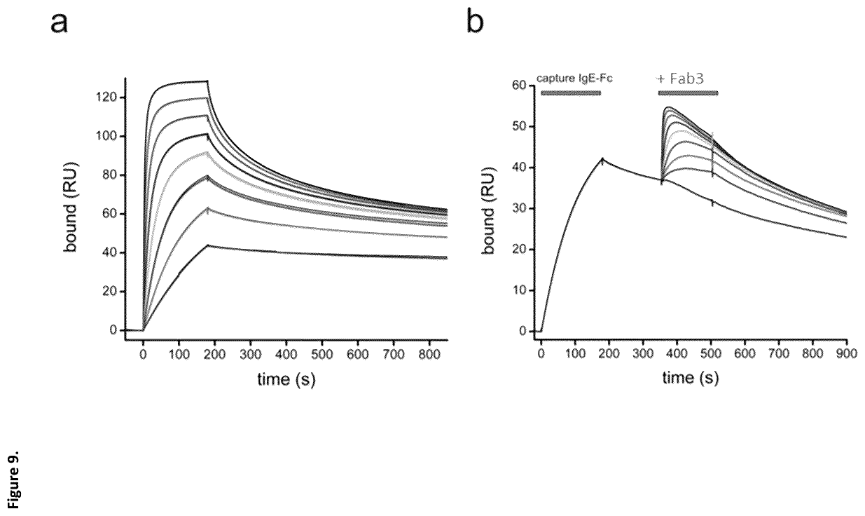

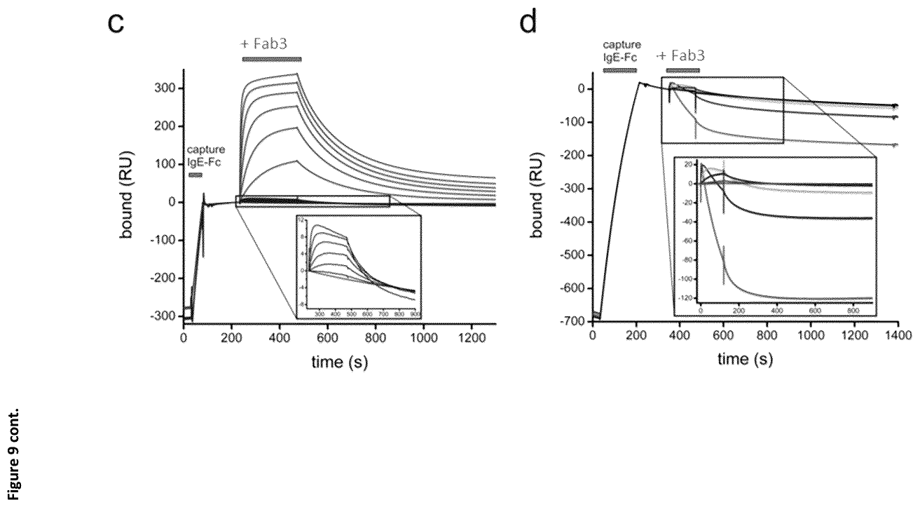

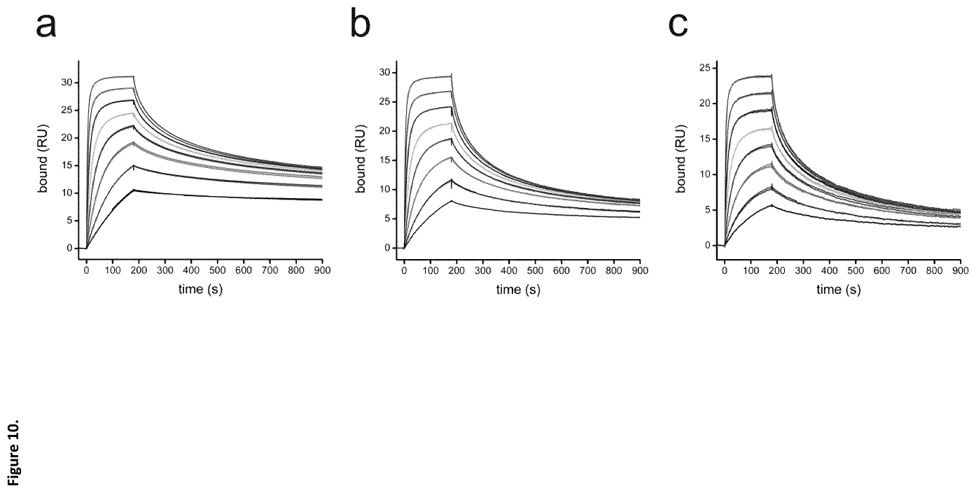

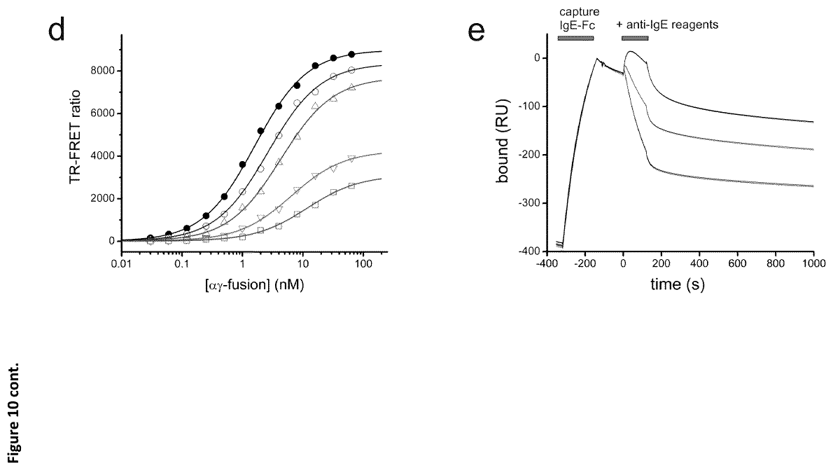

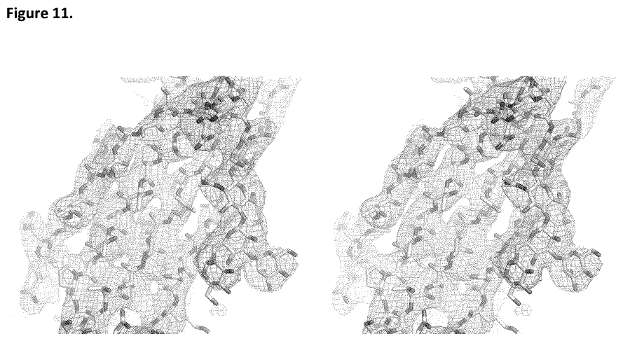

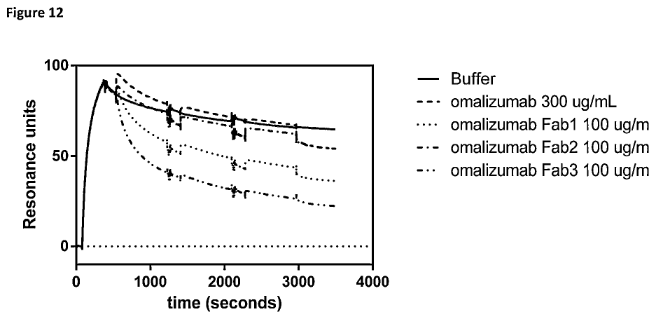

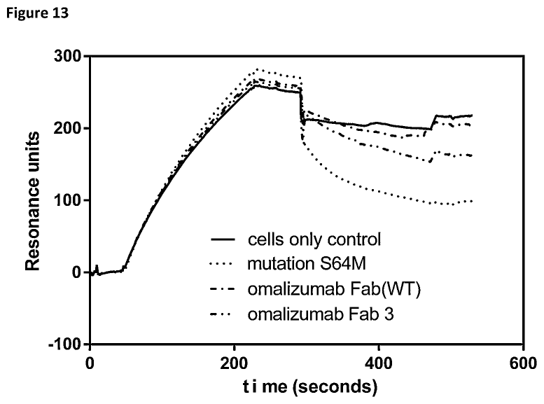

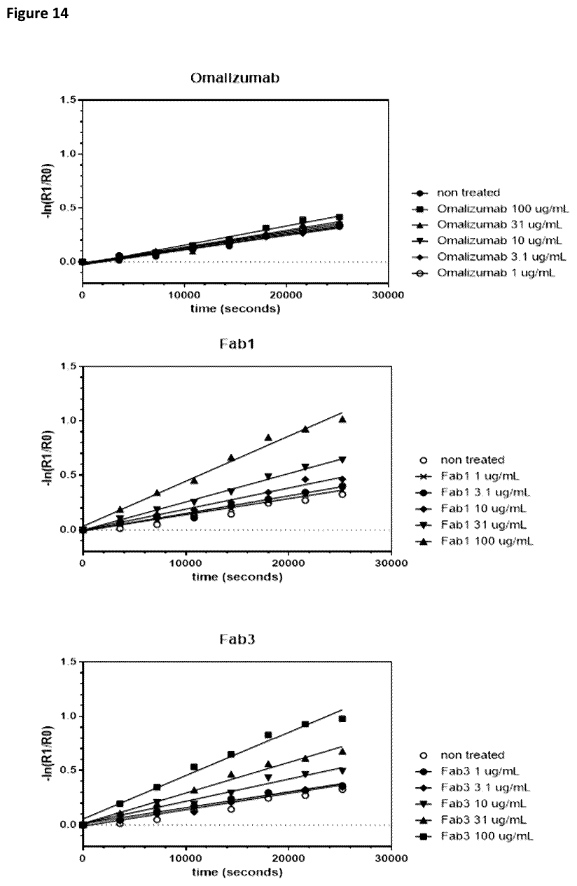

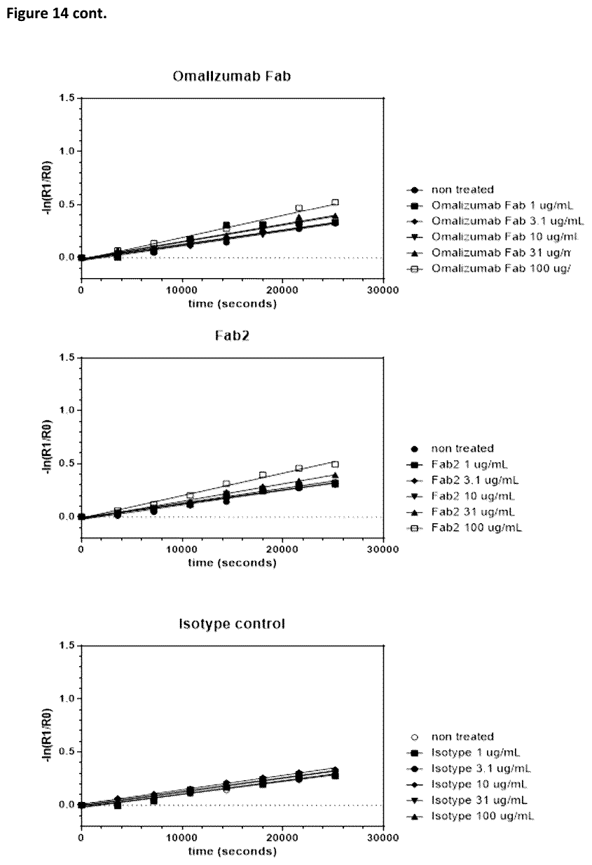

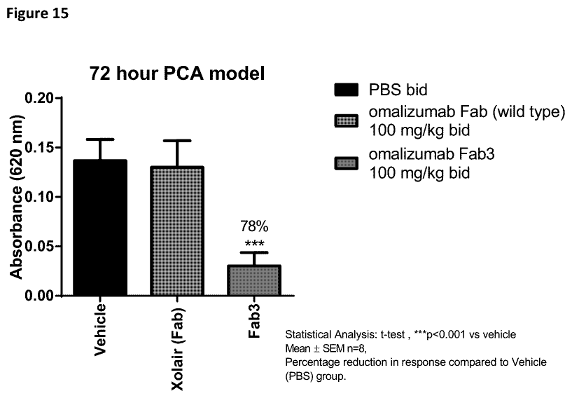

In one aspect of the present invention, the antibody or antigen binding agent binds to free and FcεRI bound human IgE. When the antibody (or antigen binding agent) of the invention binds to the FcεRI bound human IgE, it stabilizes a conformation of IgE. In such stabilized conformation, the IgE has a binding affinity for FcεRI or omalizumab (or fragments thereof) weaker than in the absence of the antibody or antigen binding agent of the present invention and wherein the FcεRI bound human IgE dissociates from FcεRI. Preferably, the IgE, upon dissociation from the FcεRI, remains bound to the antibody or antigen binding agent as described herein. As it will be shown hereinafter (for instance in Example 1 and FIG. 2), the antibody or antigen binding agent of the invention binds to the IgE in a conformation which is different from the conformation IgE has when bound to omalizumab.

Without wishing to be bound by theory, the antibody of the present invention causes the IgE to adopt a partially bent conformation (FIGS. 2C and 5C&D), hence, unbending from the free or FcεRI bound IgE structures. Such unbending compromises the ability of the IgE to bind or keep binding the FcεRI. As it will be shown in the example section, because the antibody of the present invention competes with the binding site on IgE for binding to the FcεRI, it is believed that the antibody may be capable of forming a complex with the IgE bound to the FcεRI altering the structure of the IgE when bound to the FcεRI and dissociating it from the FcεRI. Antibodies according to the present invention which possess these properties are those described herein such as antibodies or antigen binding agent comprising:
a. a heavy chain variable region comprising Seq ID No.: 1 and a light chain variable region comprising:
   i. Seq ID No.: 109; or
   ii. Seq ID No.: 113; or
   iii. Seq ID No.: 121; or
   iv. Seq ID No.: 132; or
   v. Seq ID NO. 139; or
b. Seq ID No.: 5 and
   i. Seq ID No., wherein S77 and S79 are replaced by Q;
   ii. Seq ID No.: 117 or
   iii. Seq ID No.: 125; or
   iv. Seq ID No.: 136; or
   v. Seq ID No.: 143.

Anti-IgE antibodies or antigen binding agents possessing such properties contact or contact and are specific for an epitope comprising, with reference to SEQ ID NO: 108, residues T373, W374, S375, R376, A377, S378, G379, P381, Q417, C418, R419, P426, R427, A428 of a CE3 domain and residues D278 and T281 of a Cε2 domain of human IgE.

The antibodies of the present invention possess at several positions methionine residues. Oxidation of methionine residues is among the most common protein degradation pathway. The antibodies of the present invention where methionine residues have been introduced at position S64 and S71, with reference to Seq ID No. 20, may undergo complete oxidation without affecting the ability of the antibodies to accelerate dissociation of the IgE-Fc:sFcRIα complex.

Therefore, the present invention also provides for an anti-IgE antibody, or antigen-binding agent, which comprises a heavy chain variable region which has an amino acid sequence which is Seq ID No. 1 and a light chain variable region which comprises a CDR-L2 and a FW-L3, wherein the light chain variable region has an amino acid sequence which is Seq ID No. 20, except that the CDR-L2 has an amino acid sequence which is Seq ID No. 50 and that the FW-L3 has an amino acid sequence which is Seq ID No. 131 or 138, wherein methionine residues at positions 64 and/or 71, with reference to Seq ID No. 20, are oxidised.

The present invention also provides for an anti-IgE antibody, or antigen-binding agent, which comprises a heavy chain variable region which has an amino acid sequence which is Seq ID No. 1 and a light chain variable region which has an amino acid sequence which is Seq ID No. 132 or 139, wherein methionine residues at positions 64 and/or 71, with reference to Seq ID No. 132 or 139, are oxidised.

Further methionine residues in the antibodies of the present invention may be oxidised without affecting the ability of the antibodies to accelerate dissociation of the IgE-Fc:sFcRIα complex. The invention will now be further described by way of examples with references to embodiments illustrated in the accompanying drawings.

1-103. (canceled)

104. One or more isolated DNA sequences encoding the heavy and/or light chain(s) of an anti-IgE antibody, or antigen binding agent, wherein the anti-IgE antibody, or antigen binding agent, comprises a heavy chain variable region comprising complementarity determining regions: CDR-H1, with an amino acid sequence which is SEQ ID NO: 14, CDR-H2, with an amino acid sequence which is SEQ ID NO: 16, CDR-H3, with an amino acid sequence which is SEQ ID NO: 18, and a light chain variable region comprising complementarity determining regions: CDR-L1, with an amino acid sequence which is SEQ ID NO: 29, CDR-L2, with an amino acid sequence which is SEQ ID NO: 50 or 51, CDR-L3, with an amino acid sequence which is SEQ ID NO: 33.

105. A cloning or expression vector comprising one or more DNA sequences according to claim 104.

106. A cloning or expression vector comprising one or more DNA sequences selected from Seq. ID No. 133, or Seq ID No. 135, or Seq ID No. 137, or Seq ID No. 140, or Seq. ID No. 142, or Seq ID No. 144.

107. The cloning or expression vector of claim 106 further comprising one or more DNA sequences selected from Seq. ID No. 2, Seq. ID No. 4, Seq. ID No. 6, Seq. ID No. 8, Seq. ID No. 10, or Seq. ID No. 12 or Seq. ID No. 148.

108-152. (canceled)

153. The one or more DNA sequences of claim 104, wherein the light chain variable region further comprises a framework region, FR-L3 comprising SEQ ID NO: 32 comprising one or more amino acid substitutions to strengthen the interaction of the anti-IgE antibody, or antigen binding agent, with a Cε2 domain of human IgE, and wherein:
a) the FR-L3 region is mutated at position S60 (Kabat) to M, R, K, N, Q or T;
b) the FR-L3 region is mutated at position S67 (Kabat) to M;
c) the FR-L3 region is mutated at position S77 (Kabat) to R; and/or
d) the FR-L3 region is mutated at position Q79 (Kabat) to R.

154. The one or more DNA sequences of claim 104, wherein the anti-IgE antibody, or antigen binding agent, is selected from the group consisting of a Fab fragment, modified Fab′ fragment, Fab′ fragment, F (ab′)₂ fragment, Fv, scFv, scAb, a diabody, bispecific antibody, triabody, FabFv, Fab-Fv-Fv, tribody, and a (Fab-Fv)²-Fc.

155. The one or more DNA sequences of claim 154, wherein the anti-IgE antibody, or antigen binding agent, is a Fab fragment linked directly or via a linker to a scFv that binds to a serum carrier protein, such as human serum albumin.

156. The one or more DNA sequences of claim 155, wherein the scFv comprises a heavy chain variable region and a light chain variable region, wherein the heavy chain variable region comprises a CDR-H1 comprising SEQ ID NO: 152, a CDR-H2 comprising SEQ ID NO:
153, and a CDR-H3 comprising SEQ ID NO: 154 and the light chain variable region comprises a CDR-L1 comprising SEQ ID NO: 155, a CDR-L2 comprising SEQ ID NO: 156, and a CDR-L3 comprising SEQ ID NO: 157.

157. The one or more DNA sequences of claim 156, wherein the heavy chain variable region and the light chain variable region are linked by a linker comprising SEQ ID NO: 151.

158. The one or more DNA sequences of claim 155, wherein the Fab fragment comprises a heavy chain variable region and a light chain variable region, wherein:
a) the heavy chain variable region comprises a CDR-H1 comprising SEQ ID NO: 14, a CDR-H2 comprising SEQ ID NO: 16, and a CDR-H3 comprising SEQ ID NO: 18 and the light chain variable region comprises a CDR-L1 comprising SEQ ID NO: 29, a CDR-L2 comprising SEQ ID NO: 50, a CDR-L3 comprising SEQ ID NO: 33 and a framework region FW-L3 comprising SEQ ID NO: 131 or 138; or
b) the heavy chain variable region comprises SEQ ID NO: 1 and the light chain variable region comprises SEQ ID NO: 132 or 139.

159. One or more isolated DNA sequence encoding the heavy and/or light chain(s) of an anti-IgE antibody, or antigen binding agent, wherein the anti-IgE antibody, or antigen binding agent, comprises a heavy chain variable region and a light chain variable region, wherein:
a) the heavy chain variable region comprises a CDR-H1 with an amino acid sequence which is SEQ ID NO: 14, a CDR-H2 with an amino acid sequence which is SEQ ID NO: 16 and a CDR-H3 with an amino acid sequence which is SEQ ID NO: 18 and the light chain variable region comprises CDR-L1 with an amino acid sequence which is SEQ ID NO: 29, a CDR-L2 with an amino acid sequence which is SEQ ID NO: 50, a CDR-L3 with an amino acid sequence which is SEQ ID NO: 33 and a framework region FW-L3 with an amino acid sequence which is SEQ ID NO: 131 or 138; or
b) the heavy chain variable region comprises an amino acid sequence which is SEQ ID NO: 1 and the light chain variable region comprises an amino acid sequence selected from SEQ ID NO: 132 or 139.

160. The one or more DNA sequences of claim 159 wherein the anti-IgE antibody, or antigen binding agent, further comprises a light chain constant region, wherein the light chain variable region and the light chain constant region VL-CL comprise SEQ ID NO: 136 or 143.

161. The one or more DNA sequences of claim 159, wherein the anti-IgE antibody, or antigen binding agent, further comprises a heavy chain constant region, CH1.

162. The one or more DNA sequences of claim 159, wherein the heavy chain variable region and heavy chain constant region, VH-CH1, comprise SEQ ID NO: 5.

163. A cloning or expression vector comprising the one or more DNA sequences of claim 159.


SEQUENCE LISTING (160 sequences)

SEQ ID NO: 1 - protein, 121 aa, synthetic construct [sequence omitted]

SEQ ID NO: 2 - DNA, 363 bp, synthetic construct
  misc_feature 1..363: note = V-region
  source 1..363: mol_type = other DNA
gaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagc

SEQ ID NO: 3 - protein, 139 aa, synthetic construct
  REGION 1..139: note = V-region with signal sequence
MKWVTFISLLFLFSSAYSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSS

SEQ ID NO: 4 - DNA, 417 bp, synthetic construct
  misc_feature 1..417: note = V-region with signal sequence
  source 1..417: mol_type = other DNA
atgaagtgggtcaccttcatctccctgctgtttctgttctccagcgcctactccgaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagc

SEQ ID NO: 5 - protein, 224 aa, synthetic construct
  REGION 1..224: note = v-region + gamma 1 CH1 constant region
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - DNA, 672 bp, synthetic construct
  misc_feature 1..672: note = v-region + gamma 1 CH1 constant region
  source 1..672: mol_type = other DNA
gaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 7 - protein, 242 aa, synthetic construct
  REGION 1..242: note = v-region + gamma 1 CH1 constant region with signal sequence
MKWVTFISLLFLFSSAYSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 8 - DNA, 726 bp, synthetic construct
  misc_feature 1..726: note = v-region + gamma 1 CH1 constant region with signal sequence
  source 1..726: mol_type = other DNA
atgaagtgggtcaccttcatctccctgctgtttctgttctccagcgcctactccgaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 9 - protein, 451 aa, synthetic construct
  REGION 1..451: note = v-region + gamma 1 full length constant region
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1353 bp, synthetic construct
  misc_feature 1..1353: note = v-region + gamma 1 full length constant region
  source 1..1353: mol_type = other DNA
gaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaactgctgggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggaagaaatgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 11 - protein, 469 aa, synthetic construct
  REGION 1..469: note = v-region + gamma 1 full length constant region with signal sequence
MKWVTFISLLFLFSSAYSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1407 bp, synthetic construct
  misc_feature 1..1407: note = v-region + gamma 1 full length constant region with signal sequence
  source 1..1407: mol_type = other DNA
atgaagtgggtcaccttcatctccctgctgtttctgttctccagcgcctactccgaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaactgctgggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggaagaaatgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 13 - protein, 25 aa, synthetic construct [sequence omitted]

SEQ ID NO: 14 - protein, 11 aa, synthetic construct
  REGION 1..11: note = CDRH1
GYSITSGYSWN

SEQ ID NO: 15 - protein, 14 aa, synthetic construct
  REGION 1..14: note = FR H2
WIRQAPGKGLEWVA

SEQ ID NO: 16 - protein, 16 aa, synthetic construct
  REGION 1..16: note = CDRH2
SITYDGSTNYNPSVKG

SEQ ID NO: 17 - protein, 32 aa, synthetic construct
  REGION 1..32: note = FR H3
RITISRDDSKNTFYLQMNSLRAEDTAVYYCAR

SEQ ID NO: 18 - protein, 12 aa, synthetic construct
  REGION 1..12: note = CDRH3
GSHYFGHWHFAV

SEQ ID NO: 19 - protein, 11 aa, synthetic construct
  REGION 1..11: note = FR H4
WGQGTLVTVSS

SEQ ID NO: 20 - protein, 111 aa, synthetic construct [sequence omitted]

SEQ ID NO: 21 - DNA, 333 bp, synthetic construct
  misc_feature 1..333: note = V-region light
  source 1..333: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaag

SEQ ID NO: 22 - protein, 129 aa, synthetic construct
  REGION 1..129: note = V-region light with signal sequence
MKWVTFISLLFLFSSAYSDIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIK

SEQ ID NO: 23 - DNA, 387 bp, synthetic construct
  misc_feature 1..387: note = V-region light with signal sequence
  source 1..387: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaag

SEQ ID NO: 24 - protein, 218 aa, synthetic construct
  REGION 1..218: note = V-region light + kappa constant region
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 654 bp, synthetic construct
  misc_feature 1..654: note = V-region + kappa constant region
  source 1..654: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 26 - protein, 236 aa, synthetic construct
  REGION 1..236: note = V-region + kappa constant region with signal sequence
MKWVTFISLLFLFSSAYSDIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 708 bp, synthetic construct
  misc_feature 1..708: note = V-region + kappa constant region with signal seque
  source 1..708: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28 - protein, 23 aa, synthetic construct
  REGION 1..23: note = FR L1
DIQLTQSPSSLSASVGDRVTITC

SEQ ID NO: 29 - protein, 15 aa, synthetic construct
  REGION 1..15: note = CDRL1
RASQSVDYDGDSYMN

SEQ ID NO: 30 - protein, 15 aa, synthetic construct
  REGION 1..15: note = FR L2
WYQQKPGKAPKLLIY

SEQ ID NO: 31 - protein, 7 aa, synthetic construct
  REGION 1..7: note = CDRL2
AASYLES

SEQ ID NO: 32 - protein, 32 aa, synthetic construct
  REGION 1..32: note = FR L3
GVPSRFSGSGSGTDFTLTISSLQPEDFATYYC

SEQ ID NO: 33 - protein, 9 aa, synthetic construct
  REGION 1..9: note = CDRL3
QQSHEDPYT

SEQ ID NO: 34 - protein, 10 aa, synthetic construct
  REGION 1..10: note = FR L4
FGQGTKVEIK

SEQ ID NO: 35 - protein, 111 aa, synthetic construct
  REGION 1..111: note = S60M_S77R_Q79R_v-region
DIQLTQSPSSLSASVGDRVTITCRASQSVDYDGDSYMNWYQQKPGKAPKLLIYAASYLESGVPMRFSGSGSGTDFTLTISRLRPEDFATYYCQQSHEDPYTFGQGTKVEIK

SEQ ID NO: 36 - DNA, 333 bp, synthetic construct
  misc_feature 1..333: note = S60M_S77R_Q79R_v-region
  source 1..333: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgcctatgcgcttctcgggaagcgggtcgggcaccgatttcaccctgaccatttcgagactgaggccggaggacttcgcgacttactactgccaacagtcccacgaggacccctatacgtttggccagggaaccaaggtcgaaatcaag

SEQ ID NO: 37 - protein, 129 aa, synthetic construct
  REGION 1..129: note = S60M_S77R_Q79R_v-region with signal sequence
MKWVTFISLLFLFSSAYSDIQLTQSPSSLSASVGDRVTITCRASQSVDYDGDSYMNWYQQKPGKAPKLLIYAASYLESGVPMRFSGSGSGTDFTLTISRLRPEDFATYYCQQSHEDPYTFGQGTKVEIK

SEQ ID NO: 38 - DNA, 387 bp, synthetic construct
  misc_feature 1..387: note = S60M_S77R_Q79R_v-region with signal sequence
  source 1..387: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgcctatgcgcttctcgggaagcgggtcgggcaccgatttcaccctgaccatttcgagactgaggccggaggacttcgcgacttactactgccaacagtcccacgaggacccctatacgtttggccagggaaccaaggtcgaaatcaag

SEQ ID NO: 39 - protein, 218 aa, synthetic construct
  REGION 1..218: note = S60M_S77R_Q79R_v-region + kappa constant region including L154P
DIQLTQSPSSLSASVGDRVTITCRASQSVDYDGDSYMNWYQQKPGKAPKLLIYAASYLESGVPMRFSGSGSGTDFTLTISRLRPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 40 - DNA, 654 bp, synthetic construct
  misc_feature 1..654: note = S60M_S77R_Q79R_v-region + kappa constant region including L154P
  source 1..654: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgcctatgcgcttctcgggaagcgggtcgggcaccgatttcaccctgaccatttcgagactgaggccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 41 - protein, 236 aa, synthetic construct
  REGION 1..236: note = S60M_S77R_Q79R_v-region + kappa constant region including L154P with signal sequence
MKWVTFISLLFLFSSAYSDIQLTQSPSSLSASVGDRVTITCRASQSVDYDGDSYMNWYQQKPGKAPKLLIYAASYLESGVPMRFSGSGSGTDFTLTISRLRPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 42 - DNA, 708 bp, synthetic construct
  misc_feature 1..708: note = S60M_S77R_Q79R_v-region + kappa constant region including L154P with signal sequence
  source 1..708: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgcctatgcgcttctcgggaagcgggtcgggcaccgatttcaccctgaccatttcgagactgaggccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 43 - protein, 32 aa, synthetic construct
  REGION 1..32: note = FR L3 S64M S60M
GVPMRFSGSGSGTDFTLTISSLQPEDFATYYC

SEQ ID NO: 44 - protein, 32 aa, synthetic construct
  REGION 1..32: note = FR L3 S81R S77R
GVPSRFSGSGSGTDFTLTISRLQPEDFATYYC

SEQ ID NO: 45 - protein, 32 aa, synthetic construct
  REGION 1..32: note = FR L3 Q83R Q79R
GVPSRFSGSGSGTDFTLTISSLRPEDFATYYC

SEQ ID NO: 46 - protein, 32 aa, synthetic construct
  REGION 1..32: note = FR L3 S64M S81R S60M S77R
GVPMRFSGSGSGTDFTLTISRLQPEDFATYYC

SEQ ID NO: 47 - protein, 32 aa, synthetic construct
  REGION 1..32: note = FR L3 S64M Q83R S60M Q79R
GVPMRFSGSGSGTDFTLTISSLRPEDFATYYC

SEQ ID NO: 48 - protein, 32 aa, synthetic construct
  REGION 1..32: note = FR L3 S81R Q83R S77R Q79R
GVPSRFSGSGSGTDFTLTISRLRPEDFATYYC

SEQ ID NO: 49 - protein, 32 aa, synthetic construct
  REGION 1..32: note = FR L3 S64M S81R Q83R S60M S77R Q79R
GVPMRFSGSGSGTDFTLTISRLRPEDFATYYC

SEQ ID NO: 50 - protein, 7 aa, synthetic construct
  REGION 1..7: note = CDRL2 S56D S52D
AADYLES

SEQ ID NO: 51 - protein, 7 aa, synthetic construct
  REGION 1..7: note = CDRL2 S56E S52E
AAEYLES

SEQ ID NO: 52 - protein, 32 aa, synthetic construct; identical to SEQ ID NO: 32
  REGION 1..32: note = FR L3 S71M S67M

SEQ ID NO: 53 - protein, 32 aa, synthetic construct
  REGION 1..32: note = FR L3 S64M S71M S60M S67M
GVPMRFSGSGMGTDFTLTISSLQPEDFATYYC

SEQ ID NO: 54 - protein, 32 aa, synthetic construct
  REGION 1..32: note = FR L3 S81R S71M S77R S67M
GVPSRFSGSGMGTDFTLTISRLQPEDFATYYC

SEQ ID NO: 55 - protein, 32 aa, synthetic construct
  REGION 1..32: note = FR L3 Q83R S71M Q79R S67M
GVPSRFSGSGMGTDFTLTISSLRPEDFATYYC

SEQ ID NO: 56 - protein, 32 aa, synthetic construct
  REGION 1..32: note = FR L3 S64M S81R S71M S60M S77R S67M
GVPMRFSGSGMGTDFTLTISRLQPEDFATYYC

SEQ ID NO: 57 - protein, 32 aa, synthetic construct
  REGION 1..32: note = FR L3 S64M Q83R S71M S60M Q79R S67M
GVPMRFSGSGMGTDFTLTISSLRPEDFATYYC

SEQ ID NO: 58 - protein, 32 aa, synthetic construct
  REGION 1..32: note = FR L3 S81R Q83R S71M S77R Q79R S67M
GVPSRFSGSGMGTDFTLTISRLRPEDFATYYC

SEQ ID NO: 59 - protein, 32 aa, synthetic construct
  REGION 1..32: note = FR L3 S64M S81R Q83R S71M S60M S77R Q79R S67M
GVPMRFSGSGMGTDFTLTISRLRPEDFATYYC

SEQ ID NO: 60 - protein, 32 aa, synthetic construct
  REGION 1..32: note = FR L3 S67Y S63Y
GVPSRFYGSGSGTDFTLTISSLQPEDFATYYC

SEQ ID NO: 61 - protein, 32 aa, synthetic construct
  REGION 1..32: note = FR L3 S64M S67Y S60M S63Y
GVPMRFYGSGSGTDFTLTISSLQPEDFATYYC

SEQ ID NO: 62 - protein, 32 aa, synthetic construct
  REGION 1..32: note = FR L3 S81R S67Y S77R S63Y
GVPSRFYGSGSGTDFTLTISRLQPEDFATYYC

SEQ ID NO: 63 - protein, 32 aa, synthetic construct
  REGION 1..32: note = FR L3 Q83R S67Y Q79R S63Y
GVPSRFYGSGSGTDFTLTISSLRPEDFATYYC

SEQ ID NO: 64 - protein, 32 aa, synthetic construct
  REGION 1..32: note = FR L3 S64M S81R S67Y S60M S77R S63Y
GVPMRFYGSGSGTDFTLTISRLQPEDFATYYC

SEQ ID NO: 65 - protein, 32 aa, synthetic construct
  REGION 1..32: note = FR L3 S64M Q83R S67Y S60M Q79R S63Y
GVPMRFYGSGSGTDFTLTISSLRPEDFATYYC

SEQ ID NO: 66 - protein, 32 aa, synthetic construct
  REGION 1..32: note = FR L3 S81R Q83R S67Y S77R Q79R S63Y
GVPSRFYGSGSGTDFTLTISRLRPEDFATYYC

SEQ ID NO: 67 - protein, 32 aa, synthetic construct
  REGION 1..32: note = FR L3 S64M S81R Q83R S67Y S60M S77R Q79R S63Y
GVPMRFYGSGSGTDFTLTISRLRPEDFATYYC

SEQ ID NO: 68 - protein, 32 aa, synthetic construct
  REGION 1..32: note = FR L3 S80N S76N
GVPSRFSGSGSGTDFTLTINSLQPEDFATYYC

SEQ ID NO: 69 - protein, 32 aa, synthetic construct
  REGION 1..32: note = FR L3 S64M S80N S60M S76N
GVPMRFSGSGSGTDFTLTINSLQPEDFATYYC

SEQ ID NO: 70 - protein, 32 aa, synthetic construct
  REGION 1..32: note = FR L3 S81R S80N S77R S76
GVPSRFSGSGSGTDFTLTINRLQPEDFATYYC

SEQ ID NO: 71 - protein, 32 aa, synthetic construct
  REGION 1..32: note = FR L3 Q83R S80N Q79R S76N
GVPSRFSGSGSGTDFTLTINSLRPEDFATYYC

SEQ ID NO: 72 - protein, 32 aa, synthetic construct
  REGION 1..32: note = FR L3 S64M S81R S80N S60M S77R S76N
GVPMRFSGSGSGTDFTLTINRLQPEDFATYYC

SEQ ID NO: 73 - protein, 32 aa, synthetic construct
  REGION 1..32: note = FR L3 S64M Q83R S80N S60M Q79R S76N
GVPMRFSGSGSGTDFTLTINSLRPEDFATYYC

SEQ ID NO: 74 - protein, 32 aa, synthetic construct
  REGION 1..32: note = FR L3 S81R Q83R S80N S77R Q79R S76N
GVPSRFSGSGSGTDFTLTINRLRPEDFATYYC

SEQ ID NO: 75 - protein, 32 aa, synthetic construct
  REGION 1..32: note = FR L3 S64M S81R Q83R S80N S60M S77R Q79R S76N
GVPMRFSGSGSGTDFTLTINRLRPEDFATYYC

SEQ ID NO: 76 - protein, 32 aa, synthetic construct
  REGION 1..32: note = FR L3 S80N S67Y S76N S63Y
GVPSRFYGSGSGTDFTLTINSLQPEDFATYYC

SEQ ID NO: 77 - protein, 32 aa, synthetic construct
  REGION 1..32: note = FR L3 S64M S80N S67Y S60M S76N S63Y
GVPMRFYGSGSGTDFTLTINSLQPEDFATYYC

SEQ ID NO: 78 - protein, 32 aa, synthetic construct
  REGION 1..32: note = FR L3 S81R S80N S67Y S77R S76N S63Y
GVPSRFYGSGSGTDFTLTINRLQPEDFATYYC

SEQ ID NO: 79 - protein, 32 aa, synthetic construct
  REGION 1..32: note = FR L3 Q83R S80N S67Y Q79R S76N S63Y
GVPSRFYGSGSGTDFTLTINSLRPEDFATYYC

SEQ ID NO: 80 - protein, 32 aa, synthetic construct
  REGION 1..32: note = FR L3 S64M S81R S80N S67Y S60M S77R S76N S63Y
GVPMRFYGSGSGTDFTLTINRLQPEDFATYYC

SEQ ID NO: 81 - protein, 32 aa, synthetic construct
  REGION 1..32: note = FR L3 S64M Q83R S80N S67Y S60M Q79R S76N S63Y
GVPMRFYGSGSGTDFTLTINSLRPEDFATYYC

SEQ ID NO: 82 - protein, 32 aa, synthetic construct
  REGION 1..32: note = FR L3 S81R Q83R S80N S67Y S77R Q79R S76N S63Y
GVPSRFYGSGSGTDFTLTINRLRPEDFATYYC

SEQ ID NO: 83 - protein, 32 aa, synthetic construct
  REGION 1..32: note = FR L3 S64M S81R Q83R S80N S67Y S60M S77R Q79R S76N S63Y
GVPMRFYGSGSGTDFTLTINRLRPEDFATYYC

SEQ ID NO: 84 - protein, 32 aa, synthetic construct
  REGION 1..32: note = FR L3 S67Y S71M S63Y S67M
GVPSRFYGSGMGTDFTLTISSLQPEDFATYYC

SEQ ID NO: 85 - protein, 32 aa, synthetic construct
  REGION 1..32: note = FR L3 S64M S67Y S71M S60M S63Y S67M
GVPMRFYGSGMGTDFTLTISSLQPEDFATYYC

SEQ ID NO: 86 - protein, 32 aa, synthetic construct
  REGION 1..32: note = FR L3 S81R S67Y S71M S77R S63Y S67M
GVPSRFYGSGMGTDFTLTISRLQPEDFATYYC

SEQ ID NO: 87 - protein, 32 aa, synthetic construct
  REGION 1..32: note = FR L3 Q83R S67Y S71M Q79R S63Y S67M
GVPSRFYGSGMGTDFTLTISSLRPEDFATYYC

SEQ ID NO: 88 - protein, 32 aa, synthetic construct
  REGION 1..32: note = FR L3 S64M S81R S67Y S71M S60M S77R S63Y S67M
GVPMRFYGSGMGTDFTLTISRLQPEDFATYYC

SEQ ID NO: 89 - protein, 32 aa, synthetic construct
  REGION 1..32: note = FR L3 S64M Q83R S67Y S71M S60M Q79R S63Y S67M
GVPMRFYGSGMGTDFTLTISSLRPEDFATYYC

SEQ ID NO: 90 - protein, 32 aa, synthetic construct
  REGION 1..32: note = FR L3 S81R Q83R S67Y S71M S77R Q79R S63Y S67M
GVPSRFYGSGMGTDFTLTISRLRPEDFATYYC

SEQ ID NO: 91 - protein, 32 aa, synthetic construct
  REGION 1..32: note = FR L3 S64M S81R Q83R S67Y S71M S60M S77R Q79R S63Y S67M
GVPMRFYGSGMGTDFTLTISRLRPEDFATYYC

SEQ ID NO: 92 - protein, 32 aa, synthetic construct
  REGION 1..32: note = FR L3 S80N S71M S76N S67M
GVPSRFSGSGMGTDFTLTINSLQPEDFATYYC

SEQ ID NO: 93 - protein, 32 aa, synthetic construct
  REGION 1..32: note = FR L3 S64M S80N S71M S60M S76N S67M (
GVPMRFSGSGMGTDFTLTINSLQPEDFATYYC

SEQ ID NO: 94 - protein, 32 aa, synthetic construct
  REGION 1..32: note = FR L3 S81R S80N S71M S77R S76N S67M
GVPSRFSGSGMGTDFTLTINRLQPEDFATYYC

SEQ ID NO: 95 - protein, 32 aa, synthetic construct
  REGION 1..32: note = FR L3 Q83R S80N S71M Q79R S76N S67M
GVPSRFSGSGMGTDFTLTINSLRPEDFATYYC

SEQ ID NO: 96 - protein, 32 aa, synthetic construct
  REGION 1..32: note = FR L3 S64M S81R S80N S71M S60M S77R S76N S67M
GVPMRFSGSGMGTDFTLTINRLQPEDFATYYC

SEQ ID NO: 97 - protein, 32 aa, synthetic construct
  REGION 1..32: note = FR L3 S64M Q83R S80N S71M S60M Q79R S76N S67M
GVPMRFSGSGMGTDFTLTINSLRPEDFATYYC

SEQ ID NO: 98 - protein, 32 aa, synthetic construct
  REGION 1..32: note = FR L3 S81R Q83R S80N S71M S77R Q79R S76N S67M
GVPSRFSGSGMGTDFTLTINRLRPEDFATYYC

SEQ ID NO: 99 - protein, 32 aa, synthetic construct
  REGION 1..32: note = FR L3 S64M S81R Q83R S80N S71M S60M S77R Q79R S76N S67M
GVPMRFSGSGMGTDFTLTINRLRPEDFATYYC

SEQ ID NO: 100 - protein, 32 aa, synthetic construct
  REGION 1..32: note = FR L3 S80N S67Y S71M S76N S63Y S67M
GVPSRFYGSGMGTDFTLTINSLQPEDFATYYC

SEQ ID NO: 101 - protein, 32 aa, synthetic construct
  REGION 1..32: note = FR L3 S64M S80N S67Y S71M S60M S76N S63Y S67M
GVPMRFYGSGMGTDFTLTINSLQPEDFATYYC

SEQ ID NO: 102 - protein, 32 aa, synthetic construct
  REGION 1..32: note = FR L3 S81R S80N S67Y S71M S77R S76N S63Y S67M
GVPSRFYGSGMGTDFTLTINRLQPEDFATYYC

SEQ ID NO: 103 - protein, 32 aa, synthetic construct
  REGION 1..32: note = FR L3 Q83R S80N S67Y S71M Q79R S76N S63Y S67M
GVPSRFYGSGMGTDFTLTINSLRPEDFATYYC

SEQ ID NO: 104 - protein, 32 aa, synthetic construct
  REGION 1..32: note = FR L3 S64M S81R S80N S67Y S71M S60M S77R S76N S63Y S67M
GVPMRFYGSGMGTDFTLTINRLQPEDFATYYC

SEQ ID NO: 105 - protein, 32 aa, synthetic construct
  REGION 1..32: note = FR L3 S64M Q83R S80N S67Y S71M S60M Q79R S76N S63Y S67M
GVPMRFYGSGMGTDFTLTINSLRPEDFATYYC

SEQ ID NO: 106 - protein, 32 aa, synthetic construct
  REGION 1..32: note = FR L3 S81R Q83R S80N S67Y S71M S77R Q79R S76N S63Y S67M
GVPSRFYGSGMGTDFTLTINRLRPEDFATYYC

SEQ ID NO: 107 - protein, 30 aa, synthetic construct
  REGION 1..30: note = FR L3 S64M S81R Q83R S80N S67Y S71M S60M S77R Q79R S76N S63Y S67M
PMRFYGSGMGTDFTLTINRLRPEDFATYYC

SEQ ID NO: 108 - protein, 325 aa, synthetic construct
  REGION 1..325: note = Wild-type human IgE-Fc (CE2-CE4 domains with numbering V224-K547 according to Dorrington & Bennich (1978)
VASRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 109 - protein, 111 aa, synthetic construct
  REGION 1..111: note = S77R_Q79R_v-region (Fab1)
DIQLTQSPSSLSASVGDRVTITCRASQSVDYDGDSYMNWYQQKPGKAPKLLIYAASYLESGVPSRFSGSGSGTDFTLTISRLRPEDFATYYCQQSHEDPYTFGQGTKVEIK

SEQ ID NO: 110 - DNA, 333 bp, synthetic construct
  misc_feature 1..333: note = 77R_Q79R_v-region (Fab1)
  source 1..333: mol_type = other DNA
gatattcagctgactcagagcccgagctcactctccgcttccgtgggggatagagtgaccatcacttgccgggcatcccagtcggtggactacgacggagactcctacatgaactggtaccagcagaagcccggaaaagccccaaagttgctgatctacgccgcctcctaccttgaaagcggcgtgccttcacgcttctcgggaagcgggtctggcaccgatttcaccctgaccatttcgagactgaggccggaggacttcgcgacttactactgccaacagtcccacgaggacccctatacgtttggccagggtaccaaggtcgaaatcaag

SEQ ID NO: 111 - protein, 129 aa, synthetic construct
  REGION 1..129: note = S77R_Q79R_v-region (Fab1) with signal sequence
MKWVTFISLLFLFSSAYSDIQLTQSPSSLSASVGDRVTITCRASQSVDYDGDSYMNWYQQKPGKAPKLLIYAASYLESGVPSRFSGSGSGTDFTLTISRLRPEDFATYYCQQSHEDPYTFGQGTKVEIK

SEQ ID NO: 112 - DNA, 387 bp, synthetic construct
  misc_feature 1..387: note = S77R_Q79R_v-region (Fab1) with signal sequence
  source 1..387: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtaccagcagaagcccggaaaagccccaaagttgctgatctacgccgcctcctaccttgaaagcggcgtgccttcacgcttctcgggaagcgggtctggcaccgatttcaccctgaccatttcgagactgaggccggaggacttcgcgacttactactgccaacagtcccacgaggacccctatacgtttggccagggtaccaaggtcgaaatcaag

SEQ ID NO: 113 - protein, 111 aa, synthetic construct [sequence omitted]

SEQ ID NO: 114 - DNA, 333 bp, synthetic construct; identical to SEQ ID NO: 21
  misc_feature 1..333: note = Fab2_v-region
  source 1..333: mol_type = other DNA

SEQ ID NO: 115 - protein, 129 aa, synthetic construct; identical to SEQ ID NO: 22
  REGION 1..129: note = Fab2_v-region with signal sequence

SEQ ID NO: 116 - DNA, 387 bp, synthetic construct; identical to SEQ ID NO: 23
  misc_feature 1..387: note = Fab2_v-region with signal sequence
  source 1..387: mol_type = other DNA

SEQ ID NO: 117 - protein, 218 aa, synthetic construct
  REGION 1..218: note = Fab2_v-region + kappa constant region including L154P
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 118 - DNA, 654 bp, synthetic construct
  misc_feature 1..654: note = Fab2_v-region + kappa constant region including L154P
  source 1..654: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 119 - protein, 236 aa, synthetic construct
  REGION 1..236: note = Fab2_v-region + kappa constant region including L154P with signal sequence
MKWVTFISLLFLFSSAYSDIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 120 - DNA, 708 bp, synthetic construct
  misc_feature 1..708: note = Fab2_v-region + kappa constant region including L154P with signal sequence
  source 1..708: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 121 - protein, 111 aa, synthetic construct; identical to SEQ ID NO: 109
  REGION 1..111: note = Fab3_S77R_Q79R_v-region

SEQ ID NO: 122 - DNA, 333 bp, synthetic construct; identical to SEQ ID NO: 110
  misc_feature 1..333: note = Fab3_S77R_Q79R_v-region
  source 1..333: mol_type = other DNA

SEQ ID NO: 123 - protein, 129 aa, synthetic construct; identical to SEQ ID NO: 111
  REGION 1..129: note = Fab3_S77R_Q79R_v-region with signal sequence

SEQ ID NO: 124 - DNA, 387 bp, synthetic construct; identical to SEQ ID NO: 112
  misc_feature 1..387: note = Fab3_S77R_Q79R_v-region with signal sequence
  source 1..387: mol_type = other DNA

SEQ ID NO: 125 - protein, 218 aa, synthetic construct
  REGION 1..218: note = Fab3_S77R_Q79R_v-region + kappa constant region including L154P
DIQLTQSPSSLSASVGDRVTITCRASQSVDYDGDSYMNWYQQKPGKAPKLLIYAASYLESGVPSRFSGSGSGTDFTLTISRLRPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 126 - DNA, 654 bp, synthetic construct
  misc_feature 1..654: note = Fab3_S77R_Q79R_v-region + kappa constant region including L154P
  source 1..654: mol_type = other DNA
gatattcagctgactcagagcccgagctcactctccgcttccgtgggggatagagtgaccatcacttgccgggcatcccagtcggtggactacgacggagactcctacatgaactggtaccagcagaagcccggaaaagccccaaagttgctgatctacgccgcctcctaccttgaaagcggcgtgccttcacgcttctcgggaagcgggtctggcaccgatttcaccctgaccatttcgagactgaggccggaggacttcgcgacttactactgccaacagtcccacgaggacccctatacgtttggccagggtaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 127 - protein, 236 aa, synthetic construct
  REGION 1..236: note = Fab3_S77R_Q79R_v-region + kappa constant region including L154P with signal sequence
MKWVTFISLLFLFSSAYSDIQLTQSPSSLSASVGDRVTITCRASQSVDYDGDSYMNWYQQKPGKAPKLLIYAASYLESGVPSRFSGSGSGTDFTLTISRLRPEDFATYYCQQSHEDPYTFGQGTKVEIKRTVAAPSVFIFPPSDEQLKSGTASVVCLLNNFYPREAKVQWKVDNAPQSGNSQESVTEQDSKDSTYSLSSTLTLSKADYEKHKVYACEVTHQGLSSPVTKSFNRGEC

SEQ ID NO: 128 - DNA, 708 bp, synthetic construct
  misc_feature 1..708: note = Fab3_S77R_Q79R_v-region + kappa constant region including L154P with signal sequence
  source 1..708: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtaccagcagaagcccggaaaagccccaaagttgctgatctacgccgcctcctaccttgaaagcggcgtgccttcacgcttctcgggaagcgggtctggcaccgatttcaccctgaccatttcgagactgaggccggaggacttcgcgacttactactgccaacagtcccacgaggacccctatacgtttggccagggtaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccccgcaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 129 - protein, 107 aa, synthetic construct
  REGION 1..107: note = Omalizumab_v-region
TQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIK

SEQ ID NO: 130 - DNA, 321 bp, synthetic construct
  misc_feature 1..321: note = Omalizumab_v-region
  source 1..321: mol_type = other DNA
actcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgcctcataccttgaaagcggcgtgccttcgcgcttctcgggaagcgggtcgggcaccgatttcaccctgaccatttcgtcgctgcagccggaggacttcgcgacttactactgccaacagtcccacgaggacccctatacgtttggccagggaaccaaggtcgaaatcaag

SEQ ID NO: 131 - protein, 32 aa, synthetic construct; identical to SEQ ID NO: 59
  REGION 1..32: note = FR3

SEQ ID NO: 132 - protein, 111 aa, synthetic construct
  REGION 1..111: note = S60M_S52D_S67M_S77R_Q79R_v-region
DIQLTQSPSSLSASVGDRVTITCRASQSVDYDGDSYMNWYQQKPGKAPKLLIYAADYLESGVPMRFSGSGMGTDFTLTISRLRPEDFATYYCQQSHEDPYTFGQGTKVEIK

SEQ ID NO: 133 - DNA, 333 bp, synthetic construct
  misc_feature 1..333: note = S60M_S52D_S67M_S77R_Q79R_v-region
  source 1..333: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgactaccttgaaagcggcgtgcctatgcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaag

SEQ ID NO: 134 - protein, 129 aa, synthetic construct
  REGION 1..129: note = S60M_S52D_S67M_S77R_Q79R_v-region with signal sequence
MKWVTFISLLFLFSSAYSDIQLTQSPSSLSASVGDRVTITCRASQSVDYDGDSYMNWYQQKPGKAPKLLIYAADYLESGVPMRFSGSGMGTDFTLTISRLRPEDFATYYCQQSHEDPYTFGQGTKVEIK

SEQ ID NO: 135 - DNA, 387 bp, synthetic construct
  misc_feature 1..387: note = S60M_S52D_S67M_S77R_Q79R_v-region with signal sequence
  source 1..387: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgactaccttgaaagcggcgtgcctatgcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaag

SEQ ID NO: 136 - protein, 218 aa, synthetic construct
  REGION 1..218: note = S60M_S52D_S67M_S77R_Q79R_v-region + kappa constant region
DIQLTQSPSSLSASVGDRVTITCRASQSVDYDGDSYMNWYQQKPGKAPKLLIYAADYLESGVPMRFSGSGMGTDFTLTISRLRPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 654 bp, synthetic construct
  misc_feature 1..654: note = S60M_S52D_S67M_S77R_Q79R_v-region + kappa constant region
  source 1..654: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgactaccttgaaagcggcgtgcctatgcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 138 - protein, 32 aa, synthetic construct
  REGION 1..32: note = FR3
GVPRRFSGSGMGTDFTLTISRLRPEDFATYYC

SEQ ID NO: 139 - protein, 111 aa, synthetic construct
  REGION 1..111: note = S60R_S52D_S67M_S77R_Q79R_v-region
DIQLTQSPSSLSASVGDRVTITCRASQSVDYDGDSYMNWYQQKPGKAPKLLIYAADYLESGVPRRFSGSGMGTDFTLTISRLRPEDFATYYCQQSHEDPYTFGQGTKVEIK

SEQ ID NO: 140 - DNA, 333 bp, synthetic construct
  misc_feature 1..333: note = S60R_S52D_S67M_S77R_Q79R_v-region
  source 1..333: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgattaccttgaaagcggcgtgcctcgtcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaag

SEQ ID NO: 141 - protein, 129 aa, synthetic construct
  REGION 1..129: note = S60R_S52D_S67M_S77R_Q79R_v-region with signal sequence
MKWVTFISLLFLFSSAYSDIQLTQSPSSLSASVGDRVTITCRASQSVDYDGDSYMNWYQQKPGKAPKLLIYAADYLESGVPRRFSGSGMGTDFTLTISRLRPEDFATYYCQQSHEDPYTFGQGTKVEIK

SEQ ID NO: 142 - DNA, 387 bp, synthetic construct
  misc_feature 1..387: note = S60R_S52D_S67M_S77R_Q79R_v-region with signal sequence
  source 1..387: mol_type = other DNA
atgaagtgggtcaccttcatctccctgctgtttctgttctcctccgcctactccgatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgattaccttgaaagcggcgtgcctcgtcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaag

SEQ ID NO: 143 - protein, 218 aa, synthetic construct
  REGION 1..218: note = S60R_S52D_S67M_S77R_Q79R_v-region + kappa constant region
DIQLTQSPSSLSASVGDRVTITCRASQSVDYDGDSYMNWYQQKPGKAPKLLIYAADYLESGVPRRFSGSGMGTDFTLTISRLRPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 654 bp, synthetic construct
  misc_feature 1..654: note = S60R_S52D_S67M_S77R_Q79R_v-region + kappa constant region
  source 1..654: mol_type = other DNA
gatattcagctgactcagagcccgagctcactctccgcttccgtgggggatagagtgaccatcacttgccgggcatcccagtcggtggactacgacggagactcctacatgaactggtatcagcagaagcccggaaaagccccaaagttgctgatctacgccgccgattaccttgaaagcggcgtgcctcgtcgcttctcgggaagcgggatgggcaccgatttcaccctgaccatttcgagactgaggccggaggacttcgcgacttactactgccaacagtcccacgaggacccctatacgtttggccagggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 145 - protein, 111 aa, synthetic construct
  REGION 1..111: note = S60K_S52D_S67M_S77R_Q79R_v-region
DIQLTQSPSSLSASVGDRVTITCRASQSVDYDGDSYMNWYQQKPGKAPKLLIYAADYLESGVPKRFSGSGMGTDFTLTISRLRPEDFATYYCQQSHEDPYTFGQGTKVEIK

SEQ ID NO: 146 - protein, 111 aa, synthetic construct
  REGION 1..111: note = S60Q_S52D_S67M_S77R_Q79R_v-region
DIQLTQSPSSLSASVGDRVTITCRASQSVDYDGDSYMNWYQQKPGKAPKLLIYAADYLESGVPQRFSGSGMGTDFTLTISRLRPEDFATYYCQQSHEDPYTFGQGTKVEIK

SEQ ID NO: 147 - protein, 506 aa, synthetic construct
  REGION 1..506: note = v-region + gamma 1 CH1 constant region plus linker plus CA645 gL4gH5 scFv with signal sequence
MKWVTFISLLFLFSSAYSEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 148 - DNA, 1518 bp, synthetic construct
  misc_feature 1..1518: note = v-region + gamma 1 CH1 constant region plus linker plus CA645 gL4gH5 scFv with signal sequence
  source 1..1518: mol_type = other DNA
atgaagtgggtcaccttcatctccctgctgtttctgttctccagcgcctactccgaagtgcagttggtggagtcgggtggagggctggtgcagcctggcggtagcctgaggctgtcctgtgccgtgtccggatactccattacctccggctactcgtggaactggatcagacaggctcccggaaagggacttgagtgggtggcgtccatcacctacgacggctcaaccaactataacccgtccgtgaagggccgcatcaccatttcgcgcgacgacagcaagaatactttttacctccaaatgaacagcctgcgggccgaagatactgccgtgtactactgcgcgcggggatcacattacttcgggcactggcacttcgccgtctggggacagggcaccctcgtcactgtctcgagcgcttctacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggcaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggcggagggggtagtggagggggtggctctgggggtggcggaagcggtggcgggggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 149 - protein, 11 aa, synthetic construct
  REGION 1..11: note = Linker between CH1 and CA645 gL4gH5 scFv
SGGGGTGGGGS

SEQ ID NO: 150 - protein, 253 aa, synthetic construct
  REGION 1..253: note = CA645 gL4gH5 scFv
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 151 - protein, 20 aa, synthetic construct
  REGION 1..20: note = Linker between CA645 gH5 and CA645 gL4 within scFv
GGGGSGGGGSGGGGSGGGGS

SEQ ID NO: 152 - protein, 10 aa, synthetic construct
  REGION 1..10: note = CA645 CDRH1
GIDLSNYAIN

SEQ ID NO: 153 - protein, 16 aa, synthetic construct
  REGION 1..16: note = CA645 CDRH2
IIWASGTTFYATWAKG

SEQ ID NO: 154 - protein, 13 aa, synthetic construct
  REGION 1..13: note = CA645 CDRH3
TVPGYSTAPYFDL

SEQ ID NO: 155 - protein, 12 aa, synthetic construct
  REGION 1..12: note = CA645 CDRL1
QSSPSVWSNFLS

SEQ ID NO: 156 - protein, 7 aa, synthetic construct
  REGION 1..7: note = CA645 CDRL2
EASKLTS

SEQ ID NO: 157 - protein, 10 aa, synthetic construct
  REGION 1..10: note = CA645 CDRL3
GGYSSISDTT

SEQ ID NO: 158 - protein, 111 aa, synthetic construct
  REGION 1..111: note = S60M_S52D_S67M_S77R_Q79R_S63W_S76N_v-region
DIQLTQSPSSLSASVGDRVTITCRASQSVDYDGDSYMNWYQQKPGKAPKLLIYAADYLESGVPMRFWGSGMGTDFTLTINRLRPEDFATYYCQQSHEDPYTFGQGTKVEIK

SEQ ID NO: 159 - protein, 111 aa, synthetic construct
  REGION 1..111: note = S60M_S52D_S67M_S77R_Q79R_S63Y_S76N_v-region
DIQLTQSPSSLSASVGDRVTITCRASQSVDYDGDSYMNWYQQKPGKAPKLLIYAADYLESGVPMRFYGSGMGTDFTLTINRLRPEDFATYYCQQSHEDPYTFGQGTKVEIK

SEQ ID NO: 160 - protein, 18 aa, synthetic construct
  REGION 1..18: note = Signal sequence
MKWVTFISLLFLFSSAYS